(19) United States
(12) Patent Application Publication
(10) Pub. No.: US 2025/0032390 A1
(43) Pub. Date: Jan. 30, 2025

(54) COMPOSITIONS COMPRISING CATIONIC ALPHA-GLUCAN ETHERS IN AQUEOUS POLAR ORGANIC SOLVENTS

(71) Applicant: NUTRITION & BIOSCIENCES USA 4, INC., Rochester, NY (US)

(72) Inventors: Zhengzheng HUANG, Hockessin, DE (US); Daqing ZHANG, Wilmington, DE (US)

(21) Appl. No.: 18/716,169

(22) PCT Filed: Dec. 15, 2022
(86) PCT No.: PCT/US2022/081711

§ 371 Date: Jun. 4, 2024

Related U.S. Application Data
(60) Provisional application No. 63/290,473, filed on Dec. 16, 2021.

(51) Int. Cl. A61K 8/73; A61Q 5/06; C09D 7/20; C09D 105/00

(52) U.S. Cl. CPC A61K 8/73; A61Q 5/06; C09D 7/20; C09D 105/00

(57) ABSTRACT

Compositions are disclosed herein comprising at least a solvent and an ether derivative of an alpha-glucan. Typically, (i) at least about 40% of the glycosidic linkages of the alpha-glucan are alpha-1,6 linkages, (ii) the alpha-glucan has a weight-average molecular weight of about 1 kDa to about 2000 kDa, (iii) the alpha-glucan has a degree of substitution of about 0.01 to about 3.0 with at least one positively charged organic group that is ether-linked to the alpha-glucan, and (iv) the solvent comprises water and at least about 40% (v/v) of a polar organic solvent. The ether derivative is dissolved and/or dispersed in the solvent. Methods are further disclosed for preparing these compositions, as well as various applications of using them.

This application claims the benefit of U.S. Provisional Appl. No. 63/290,473 (filed Dec. 16, 2021), which is incorporated herein by reference in its entirety.

### FIELD

The present disclosure is in the field of polysaccharide derivatives. For example, the disclosure pertains to cationic alpha-glucan ether derivatives such as cationic alpha-1,6-glucan ether derivatives, and use thereof in various applications.

### BACKGROUND

Driven by a desire to find new structural polysaccharides using enzymatic syntheses or genetic engineering of microorganisms, researchers have discovered oligosaccharides and polysaccharides that are biodegradable and that can be made economically from renewably-sourced feedstocks. Further work has shown that such polysaccharides can be chemically modified (derivatized) to have additional utilities in areas such as personal care, household care, industrial care, pharmaceuticals and food. For example, ethers and esters of alpha-glucan comprising alpha-1,3 glycosidic linkages have been disclosed to have various applications (e.g., U.S. Patent Appl. Publ. Nos. 2016/0304629, 2016/0311935, 2017/0204232, 2014/0187767, 2020/0308371). Various derivatives of alpha-glucan comprising alpha-1,6 glycosidic linkages, and applications for use thereof, have also been disclosed (e.g., U.S. Patent Appl. Publ. Nos. 2018/0312781, 2018/0237816, 2018/0282385).

Cationic alpha-glucan ethers exhibit various beneficial effects such as surface deposition and modification. Aqueous products that take advantage of these effects typically contain solvent that is mostly or completely water, which allows for adequate solvation of cationic alpha-glucan ether ingredients. However, aqueous products having an elevated polar organic solvent (e.g., alcohol) content typically cannot carry cationic alpha-glucan ethers in a stable manner, and so cannot deliver the benefits afforded by these ether compounds. Compositions are disclosed herein that address this issue.

### SUMMARY

In one embodiment, the present disclosure concerns a composition comprising at least a solvent and an ether derivative of an alpha-glucan, wherein (i) at least about 40% of the glycosidic linkages of the alpha-glucan are alpha-1,6 linkages, (ii) the alpha-glucan has a weight-average molecular weight (Mw) of about 1 kDa to about 2000 kDa, (iii) the alpha-glucan has a degree of substitution (DoS) of about 0.01 to about 3.0 with at least one positively charged organic group that is ether-linked to the alpha-glucan, and (iv) the solvent comprises water and at least about 40% (v/v) of a polar organic solvent; wherein the ether derivative is dissolved and/or dispersed in the solvent.

In another embodiment, the present disclosure concerns a method of producing the foregoing composition. Such a method comprises: mixing the solvent herein and the ether derivative together, thereby producing the composition.

In another embodiment, the present disclosure concerns a method of producing a film or coating. Such a method comprises: (a) providing the foregoing composition, (b) contacting the composition with a surface, and (c) removing at least about 95% by weight of the solvent to form a film or coating on the surface.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like. The numerical values of the various ranges in the present disclosure, unless expressly indicated otherwise, are stated as approximations as though the minimum and maximum values within the stated ranges were both proceeded by the word “about”. In this manner, slight variations above and below the stated ranges can typically be used to achieve substantially the same results as values within the ranges. Also, the disclosure of these ranges is intended as a continuous range including each and every value between the minimum and maximum values.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

It is to be appreciated that certain features of the present disclosure, which are, for clarity, described above and below in the context of aspects/embodiments, may also be provided in combination in a single element. Conversely, various features of the disclosure that are, for brevity, described in the context of a single aspect/embodiment, can also be provided separately or in any sub-combination.

A “glucan” herein is a type of polysaccharide that is a polymer of glucose (polyglucose). A glucan can be comprised of, for example, about, or at least about, 90%, 91%, 92%, 93%, 94%, 95%, 96%, 97%, 98%, 99%, or 100% by weight glucose monomeric units. An example of a glucan herein is alpha-glucan.

The terms “alpha-glucan”, “alpha-glucan polymer” and the like are used interchangeably herein. An alpha-glucan is a polymer comprising glucose monomeric units linked together by alpha-glycosidic linkages. In typical aspects, the glycosidic linkages of an alpha-glucan herein are about, or at least about, 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. An example of an alpha-glucan polymer herein is alpha-1,6-glucan.

The term “saccharide” and other like terms herein refer to monosaccharides and/or disaccharides/oligosaccharides, unless otherwise noted. A “disaccharide” herein refers to a carbohydrate having two monosaccharides joined by a glycosidic linkage. An “oligosaccharide” herein can refer to a carbohydrate having 3 to 15 monosaccharides, for example, joined by glycosidic linkages. An oligosaccharide can also be referred to as an “oligomer”. Monosaccharides (e.g., glucose and/or fructose) comprised within disaccharides/oligosaccharides can be referred to as “monomeric units”, “monosaccharide units”, or other like terms.

The terms “alpha-1,6-glucan”, “poly alpha-1,6-glucan”, “alpha-1,6-glucan polymer”, “dextran”, and the like herein refer to a water-soluble alpha-glucan comprising glucose monomeric units linked together by glycosidic linkages, wherein at least about 40% of the glycosidic linkages are alpha-1,6. Alpha-1,6-glucan in some aspects comprises about, or at least about, 90%, 95%, or 100% alpha-1,6 glycosidic linkages. Other linkages that can be present in alpha-1,6-glucan include alpha-1,2, alpha-1,3, and/or alpha-1,4 linkages.

An “alpha-1,2 branch” (and like terms) as referred to herein typically comprises a glucose that is alpha-1,2-linked to a dextran backbone; thus, an alpha-1,2 branch herein can also be referred to as an alpha-1,2,6 linkage. An alpha-1,2 branch herein typically has one glucose group (can optionally be referred to as a pendant glucose).

An “alpha-1,3 branch” (and like terms) as referred to herein typically comprises a glucose that is alpha-1,3-linked to a dextran backbone; thus, an alpha-1,3 branch herein can also be referred to as an alpha-1,3,6 linkage. An alpha-1,3 branch herein typically has one glucose group (can optionally be referred to as a pendant glucose).

The percent branching in an alpha-glucan herein refers to that percentage of all the linkages in the alpha-glucan that represent branch points. For example, the percent of alpha-1,2 branching in an alpha-glucan herein refers to that percentage of all the linkages in the glucan that represent alpha-1,2 branch points. Except as otherwise noted, linkage percentages disclosed herein are based on the total linkages of an alpha-glucan, or the portion of an alpha-glucan for which a disclosure specifically regards.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages), 1,3-alpha-D-glycosidic linkages (herein also referred to as “alpha-1,3” linkages), 1,4-alpha-D-glycosidic linkages (herein also referred to as “alpha-1,4” linkages), and 1,2-alpha-D-glycosidic linkages (herein also referred to as “alpha-1,2” linkages).

The glycosidic linkage profile of an alpha-glucan or derivative thereof can be determined using any method known in the art. For example, a linkage profile can be determined using methods using nuclear magnetic resonance (NMR) spectroscopy (e.g., ¹³C NMR and/or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The “molecular weight” of an alpha-glucan or alpha-glucan derivative herein can be represented as weight-average molecular weight (Mw) or number-average molecular weight (Mn), the units of which are in Daltons (Da) or grams/mole. Alternatively, molecular weight can be represented as DPw (weight average degree of polymerization) or DPn (number average degree of polymerization). The molecular weight of smaller alpha-glucan polymers such as oligosaccharides can optionally be provided as “DP” (degree of polymerization), which simply refers to the number of monomers comprised within the alpha-glucan; “DP” can also characterize the molecular weight of a polymer on an individual molecule basis. Various means are known in the art for calculating these various molecular weight measurements such as with high-pressure liquid chromatography (HPLC), size exclusion chromatography (SEC), or gel permeation chromatography (GPC).

As used herein, Mw can be calculated as Mw=ΣNiMi²/ΣNiMi; where Mi is the molecular weight of an individual chain i and Ni is the number of chains of that molecular weight. Besides SEC, the Mw of a polymer can be determined by other techniques such as static light scattering, mass spectrometry, MALDI-TOF (matrix-assisted laser desorption/ionization time-of-flight), small angle X-ray or neutron scattering, or ultracentrifugation. As used herein, Mn can be calculated as Mn=ΣNiMi/ΣNi where Mi is the molecular weight of a chain i and Ni is the number of chains of that molecular weight. Besides SEC, the Mn of a polymer can be determined by various colligative property methods such as vapor pressure osmometry, end-group determination by spectroscopic methods such as proton NMR, proton FTIR, or UV-Vis. As used herein, DPn and DPw can be calculated from Mw and Mn, respectively, by dividing them by molar mass of the one monomer unit M₁. In the case of unsubstituted glucan polymer, M₁=162. In the case of a substituted (derivatized) glucan polymer, M₁=162+M_{f}×DoS, where M_{f} is molar mass of the substituting group, and DoS is degree of substitution (average number of substituted groups per one glucose unit of the glucan polymer).

An “alpha-glucan derivative” (and like terms) herein typically refers to an alpha-glucan that has been substituted with at least one type of organic group. The degree of substitution (DoS) of an alpha-glucan derivative can be up to about 3.0 (e.g., about 0.001 to about 3.0) in some aspects. An organic group herein that is an ether group is linked to an alpha-glucan derivative via ether linkage. A precursor of an alpha-glucan derivative herein typically refers to the non-derivatized alpha-glucan used to make the derivative (can also be referred to as the alpha-glucan portion of the derivative). An organic group herein typically is positively charged (cationic); generally, such charge can be as it exists when the organic group is in an aqueous composition herein, further taking into account the pH of the aqueous composition (in some aspects, the pH can be 4-10, 5-9, 6-8, or any pH as disclosed herein). The term “degree of substitution” (DoS, or DS) as used herein refers to the average number of hydroxyl groups that are substituted with one or more types of organic group (e.g., via an ether linkage) in each monomeric unit of an alpha-glucan derivative. The DoS of an alpha-glucan derivative herein can be stated with reference to the DoS of a specific substituent, or the overall DoS, which is the sum of the DoS values of different substituent types (e.g., if a mixed ether). Unless otherwise disclosed, when DoS is not stated with reference to a specific substituent type, the overall DoS is meant.

Terms used herein regarding “ethers” (e.g., alpha-glucan ether derivative) can be as disclosed, for example, in U.S. Patent Appl. Publ. Nos. 2016/0311935, 2018/0237816, or 2020/0002646, or Int. Pat. Appl. Publ. No. WO2021/257786 (Appl. No. PCT/US2021/37756), which are each incorporated herein by reference. The terms “alpha-glucan ether derivative”, “alpha-glucan ether compound”, “alpha-glucan ether”, and the like are used interchangeably herein. An alpha-glucan ether derivative herein is alpha-glucan that has been etherified with one or more organic groups (e.g., charged organic group such as cationic group) such that the derivative has a DoS with one or more organic groups of up to about 3.0. An alpha-glucan ether derivative is termed an “ether” herein by virtue of comprising the substructure —C_{G}—O—C—, where “—CO—” represents a carbon atom of a monomeric unit (typically glucose) of the alpha-glucan ether derivative (where such carbon atom was bonded to a hydroxyl group [—OH] in the alpha-glucan precursor of the ether), and where “—C—” is a carbon atom of an organic group.

An organic group can refer to a “positively charged organic group”. A positively charged organic group as used herein refers to one or more carbons (e.g., “carbon chain”) that has one or more hydrogens substituted with another atom or functional group (i.e., a “substituted alkyl group”), where one or more of the substitutions is with a positively charged group. Where a positively charged organic group has a substitution in addition to a substitution with a positively charged group, such additional substitution may be with one or more hydroxyl groups, oxygen atoms (thereby forming an aldehyde or ketone group), alkyl groups, and/or additional positively charged groups. A positively charged organic group has a net positive charge since it comprises one or more positively charged groups. The terms “positively charged group”, “positively charged ionic group”, “cationic group” and the like are used interchangeably herein. A positively charged group comprises a cation (a positively charged ion). Examples of positively charged groups include substituted ammonium groups, carbocation groups and acyl cation groups.

The terms “substituted ammonium”, “substituted ammonium group”, “substituted ammonium ion”, “substituted ammonium cation” and the like are used interchangeably herein. A substituted ammonium group herein comprises Structure I:

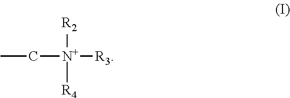

R₂, R₃ and R₄ in Structure I each independently represent a hydrogen atom or an alkyl, aryl, cycloalkyl, aralkyl, or alkaryl group. The positioning of R₂, R₃ and R₄ in Structure I is generally of no particular importance and not intended to invoke any particular stereochemistry. The carbon atom (C) in Structure I is part of one or more carbons (e.g., “carbon chain”) of the positively charged organic group. The carbon atom is either directly ether-linked to a glucose monomeric unit of an alpha-glucan herein, or is part of a chain of two or more carbon atoms that is ether-linked to the glucose monomeric unit. The carbon atom (C) in Structure I can be —CH₂—, —CH—(where an H is substituted with another group such as a hydroxy group), or —C—(where both H's are substituted).

A substituted ammonium group herein can be a “primary ammonium group”, “secondary ammonium group”, “tertiary ammonium group”, or “quaternary ammonium” group, depending on the composition of R₂, R₃ and R₄ in Structure I. A primary ammonium group herein refers to Structure I in which each of R₂, R₃ and R₄ is a hydrogen atom (i.e., —C—NH₃⁺). A secondary ammonium group herein refers to Structure I in which each of R₂ and R₃ is a hydrogen atom and R₄ is an alkyl, aryl, cycloalkyl, aralkyl, or alkaryl group. A tertiary ammonium group herein refers to Structure I in which R₂ is a hydrogen atom and each of R₃ and R₄ is an alkyl, aryl, cycloalkyl, aralkyl, or alkaryl group. Assignment herein of R₂, R₃ and R₄ is completely arbitrary. A quaternary ammonium group herein refers to Structure I in which each of R₂, R₃ and R₄ is independently an alkyl, aryl, cycloalkyl, aralkyl, or alkaryl group (i.e., none of R₂, R₃ and R₄ is a hydrogen atom). It would be understood that a fourth member (i.e., R₁) implied by the above nomenclature is the one or more carbons (e.g., chain) of the positively charged organic group that is ether-linked to a glucose monomeric unit of the alpha-glucan.

Examples of substituted ammonium alpha-glucan ethers herein comprise a hydroxypropyl group that links the ammonium group to the alpha-glucan. The positively charged organic group of such an ether compound can be represented as Structure II:

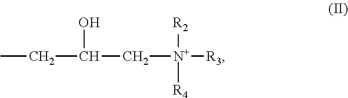

where each of R₂, R₃ and R₄ is as described above for either a primary, secondary, tertiary, or quaternary ammonium group.

The terms “aqueous liquid”, “aqueous fluid”, “aqueous conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., suspensions, colloidal dispersions) and emulsions, for example. In some embodiments, the pH of an aqueous composition is between ˜2 and ˜11 (e.g., between ˜4 and ˜9).

As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. The terms “dispersant” and “dispersion agent” are used interchangeably herein to refer to a material that promotes the formation and/or stabilization of a dispersion. “Dispersing” herein refers to the act of preparing a dispersion of a material in an aqueous liquid. As used herein, the term “latex” (and like terms) refers to a dispersion of one or more types of polymer particles in water or aqueous solution. In some aspects, a latex is an emulsion that comprises dispersed particles. An “emulsion” herein is a dispersion of minute droplets of one liquid in another liquid in which the droplets are not soluble or miscible (e.g., a non-polar substance such as oil or other organic liquid such as an alkane, in a polar liquid such as water or aqueous solution).

An alpha-glucan ether derivative herein that is “soluble”, “aqueous-soluble”, “water-soluble” (and like terms) dissolves (or appreciably dissolves) in water or other aqueous conditions herein such as those further comprising a polar organic solvent, optionally where the aqueous conditions are further characterized to have a pH of 4-9 (e.g., pH 6-8) and/or temperature of about 1 to 130° C. (e.g., 20-25° C.). In contrast, an alpha-glucan derivative that is “insoluble”, “aqueous-insoluble”, “water-insoluble” and the like does not dissolve under these conditions. In some aspects, less than 1.0 gram (e.g., no detectable amount) of an aqueous-insoluble alpha-glucan ether derivative dissolves in 1000 milliliters of such aqueous conditions (e.g., water at 23° C.).

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pa-s), for example. A centipoise is one one-hundredth of a poise; one poise is equal to 0.100 kg·m⁻¹·s⁻¹.

An alpha-glucan ether derivative herein that is dispersed in an aqueous composition herein can be stably dispersed, for example. The “stability” (or the quality of being “stable”) of a dispersion or emulsion herein can be, for example, the ability of dispersed particles of a dispersion, or liquid droplets dispersed in another liquid (emulsion), to remain dispersed (e.g., about, or at least about, 70, 75, 80, 85, 90, 95, 96, 97, 98, 99, or 100 wt % of the particles of the dispersion or liquid droplets of the emulsion are in a dispersed state) for a period of about, or at least about, 2, 4, 6, 9, 12, 18, 24, 30, or 36 months following initial preparation of the dispersion or emulsion. A stable dispersion or emulsion can resist total creaming, sedimentation, flocculation, and/or coalescence of dispersed/emulsified material. Dispersed alpha-glucan ether derivative particles can provide stability to an emulsion in some aspects.

The terms “polar organic solvent” and “water-miscible organic solvent” (and like terms) are used interchangeably herein. A polar organic solvent can be dissolved in water or an aqueous solution. Thus, a polar organic solvent does not separate out into a different phase when added to water or an aqueous solution. A polar organic solvent contains carbon and at least one heteroatom (i.e., non-carbon or -hydrogen atom) such as oxygen, nitrogen, sulfur, or phosphorous. This contrasts with non-polar organic solvents, which generally comprise only carbon and hydrogen atoms. A polar organic solvent typically has a dielectric constant greater than about 4. A polar organic solvent contains dipoles due to polar bonds.

The term “aprotic polar organic solvent” (and like terms) herein refers to a polar organic solvent that does not have suitably labile hydrogen atoms that can form hydrogen bonds. An aprotic polar organic solvent does not contain hydrogen atoms bonded to an atom with electronegative character; e.g., there are no O—H, N—H, or S—H bonds.

The term “protic polar organic solvent” (and like terms) herein refers to a polar organic solvent that has one or more suitably labile hydrogen atoms that can form hydrogen bonds. A protic polar organic solvent generally contains hydrogen atoms bonded to an atom with electronegative character; e.g., there are one or more O—H, N—H, and/or S—H bonds.

The terms “fiber”, “fibers” and the like herein can refer to staple fibers (staple length fibers) or continuous fibers, in some aspects. Fibers herein can comprise alpha-1,3-glucan, natural fiber (e.g., cellulose, cotton, wool, silk), or synthetic fiber (e.g., polyester), or any other type of material disclosed herein that can form a fiber. Fibers can be in a fiber-containing article/material/composition, for example, such as a woven or non-woven product.

The term “woven product” and like terms herein refer to a product formed by weaving, braiding, interlacing, or otherwise intertwining threads or fibers in an organized, consistent, and/or repeating manner.

The terms “non-woven”, “non-woven product”, “non-woven web” and the like herein refer to a web of individual fibers or filaments that are interlaid, typically in a random or unidentifiable manner. This contrasts with a knitted or woven fabric, which has an identifiable network of fibers or filaments. In some aspects, a non-woven product comprises a non-woven web that is bound or attached to another material such as a substrate or backing. A non-woven in some aspects can further contain a binder or adhesive (strengthening agent) that binds adjacent non-woven fibers together. A non-woven binder or adhesive agent can be applied to the non-woven in the form of a dispersion/latex, solution, or solid, for example, and then the treated non-woven is typically dried.

The terms “fabric”, “textile”, “cloth” and the like are used interchangeably herein to refer to a woven material having a network of natural and/or artificial fibers. Such fibers can be in the form of thread or yarn, for example. However, in some aspects, a fabric can comprise non-woven fibers.

The term “paint” (and like terms) herein is a type of coating composition that is a dispersion of a pigment in a suitable liquid (e.g., aqueous liquid) that can be used to form an adherent coating when spread on a surface in a thin coat. Paint as applied to a surface can provide coloration/decoration, protection, and/or treatment (e.g., primer) to the surface. A paint in some aspects, by virtue of further comprising dispersed particles, can optionally be characterized as a latex or latex paint.

The terms “film”, “sheet” and like terms herein refer to a generally thin, continuous material. A film can be comprised as a layer or coating on a material, or can be alone (e.g., not attached to a material surface; free-standing). A “coating” (and like terms) as used herein refers to a layer covering a surface of a material. The term “uniform thickness” as used to characterize a film or coating herein can refer to a contiguous area that (i) is at least 20% of the total film/coating area, and (ii) has a standard deviation of thickness of less than about 50 nm, for example. The term “continuous layer” means a layer of a composition applied to at least a portion of a substrate, wherein a dried layer of the composition covers ≥99% of the surface to which it has been applied and having less than 1% voids in the layer that expose the substrate surface. The ≥99% of the surface to which the layer has been applied excludes any area of the substrate to which the layer has not been applied. A coating herein can make a continuous layer in some aspects. A coating composition (and like terms) herein refers to all the solid components that form a layer on a substrate, such as an alpha-glucan ether derivative herein and, optionally, pigment, surfactant, dispersing agent, binder, crosslinking agent, and/or other additives.

The term “household care product” and like terms typically refer to products, goods and services relating to the treatment, cleaning, caring and/or conditioning of a home and/or its contents. The foregoing include, for example, chemicals, compositions, products, or combinations thereof having application in such care.

A “fabric care composition” and like terms refer to any composition suitable for treating fabric in some manner. Examples of such a composition include laundry detergents and fabric softeners, which are examples of laundry care compositions.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The terms “heavy duty detergent”, “all-purpose detergent” and the like are used interchangeably herein to refer to a detergent useful for regular washing of white and colored textiles at any temperature. The terms “low duty detergent”, “fine fabric detergent” and the like are used interchangeably herein to refer to a detergent useful for the care of delicate fabrics such as viscose, wool, silk, microfiber or other fabric requiring special care. “Special care” can include conditions of using excess water, low agitation, and/or no bleach, for example.

The terms “fabric softener”, “fabric conditioner” and the like herein refer to compositions, such as in liquid or solid form, that deposit lubricants and/or other surface-modifying ingredients onto fabric to, for example, help maintain softness of the fabric and/or provide other beneficial features to fabric (e.g., lubricity, anti-static, anti-cling, and/or anti-wrinkling). A fabric softener herein typically is applied to fabric following fabric washing with a laundry detergent, usually while rinsing the fabric.

The term “personal care product” and like terms typically refer to products, goods and services relating to the treatment, cleaning, cleansing, caring, or conditioning of a person. The foregoing include, for example, chemicals, compositions, products, or combinations thereof having application in such care.

An “oral care composition” herein is any composition suitable for treating a soft or hard surface in the oral cavity such as dental (teeth) and/or gum surfaces.

The terms “ingestible product”, “ingestible composition” and the like refer to any substance that, either alone or together with another substance, may be taken orally (i.e., by mouth), whether intended for consumption or not. Thus, an ingestible product includes food/beverage products. “Food/beverage products” refer to any edible product intended for consumption (e.g., for nutritional purposes) by humans or animals, including solids, semi-solids, or liquids. A “food” herein can optionally be referred to as a “foodstuff”, “food product”, or other like term, for example. “Non-edible products” (“non-edible compositions”) refer to any composition that can be taken by the mouth for purposes other than food or beverage consumption. Examples of non-edible products herein include supplements, nutraceuticals, functional food products, pharmaceutical products, oral care products (e.g., dentifrices, mouthwashes), and cosmetic products such as sweetened lip balms. A “pharmaceutical product”, “medicine”, “medication”, “drug” or like term herein refers to a composition used to treat disease or injury, and can be administered enterally or parenterally.

The term “medical product” and like terms typically refer to products, goods and services relating to the diagnosis, treatment, and/or care of patients.

The term “industrial product” and like terms typically refer to products, goods and services used in industrial and/or institutional settings, but typically not by individual consumers.

The terms “sequence identity”, “identity” and the like as used herein with respect to a polypeptide amino acid sequence (e.g., that of a glucosyltransferase) are as defined and determined in U.S. Patent Appl. Publ. No. 2017/0002336, which is incorporated herein by reference.

Various polypeptide amino acid sequences are disclosed herein as features of certain embodiments. Variants of these sequences that are at least about 70-85%, 85-90%, or 90%-95% identical to the sequences disclosed herein can be used or referenced. Alternatively, a variant amino acid sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity with a sequence disclosed herein. The variant amino acid sequence has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, or 99% of the function/activity of the disclosed sequence.

A composition herein that is “dry” or “dried” typically has less than 6, 5, 4, 3, 2, 1, 0.5, or 0.1 wt % water comprised therein.

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The term “isolated” means a substance (or process) in a form or environment that does not occur in nature. A non-limiting example of an isolated substance includes any glucan ether derivative disclosed herein, or a composition comprising such a derivative. It is believed that the embodiments disclosed herein are synthetic/man-made (could not have been made or practiced except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some aspects of the present disclosure concern a composition comprising at least a solvent and an ether derivative of an alpha-glucan (i.e., an alpha-glucan ether), wherein
(i) at least about 40% of the glycosidic linkages of the alpha-glucan are alpha-1,6 linkages,
(ii) the alpha-glucan has a weight-average molecular weight (Mw) of about 1 kDa to about 2000 kDa,
(iii) the alpha-glucan has a degree of substitution (DoS) of about 0.01 (or about 0.001) to about 3.0 with at least one positively charged organic group that is ether-linked to the alpha-glucan, and
(iv) the solvent comprises water and at least about 40% (v/v) (or w/w) of a polar organic solvent;
   wherein the ether derivative typically is dissolved and/or dispersed in the solvent.

In some aspects, an alpha-glucan ether comprises about, or at least about, 40%, 50%, 60%, 70%, 80%, 85%, 90%, 91%, 92%, 93%, 94%, 95%, 96%, 97%, 98%, 99%, 99.5%, or 100% alpha-1,6 glycosidic linkages (i.e., the ether is an alpha-1,6-glucan ether, or dextran ether). In some aspects, a substantially linear dextran ether can comprise 5%, 4%, 3%, 2%, 1%, 0.5% or less glycosidic branches (a linear dextran ether has 100% alpha-1,6 linkages). If present, glycosidic branches from a dextran ether are typically short, being one (pendant), two, or three glucose monomers in length. In some aspects, a dextran ether can comprise about, or less than about, 50%, 40%, 30%, 20%, 15%, 10%, 9%, 8%, 7%, 6%, 5%, 4%, 3%, 2%, 1%, 0.5%, or 0% alpha-1,4, alpha-1,3 and/or alpha-1,2 glycosidic linkages. Typically, such linkages exist entirely, or almost entirely, as branch points from alpha-1,6-glucan.

The dextran portion of a dextran ether derivative herein can have alpha-1,2, alpha-1,3, and/or alpha-1,4 branches, for example. In some aspects, about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 2-30%, 2-25%, 2-20%, 2-15%, 2-10%, 5-30%, 5-25%, 5-20%, 5-15%, 5-10%, 10-30%, 10-25%, 10-20%, 10-15%, 15-30%, 15-25%, 15-20%, 15-15%, 17-23%, 18-22%, 19-21%, 35-45%, 37-43%, 38-42%, or 39-41% of all the glycosidic linkages of a branched dextran ether are alpha-1,2, alpha-1,3, and/or alpha-1,4 glycosidic branch linkages. Such branches typically are mostly (>90% or >95%), or all (100%), a single glucose monomer in length. In some aspects, dextran with alpha-1,2-branching can be produced enzymatically according to the procedures in U.S. Patent Appl. Publ. Nos. 2017/0218093 or 2018/0282385 (both incorporated herein by reference) where, for example, an alpha-1,2-branching enzyme such as GTFJ18T1 or GTF9905 can be added during or after the production of the dextran. In some aspects, any other enzyme known to produce alpha-1,2-branching can be used. Dextran with alpha-1,3-branching can be prepared, for example, as disclosed in Vuillemin et al. (2016, J. Biol Chem. 291:7687-7702) or Int. Patent Appl. Publ. No. WO2021/007264, which are incorporated herein by reference.

The dextran portion of a dextran ether derivative herein can have a weight-average molecular weight (Mw) of about, at least about, or less than about, 1, 5, 7.5, 10, 12.5, 15, 20, 30, 40, 50, 60, 70, 80, 90, 100, 150, 200, 250, 300, 400, 500, 600, 700, 800, 900, 1000, 1250, 1500, 1750, 2000, 1-2000, 1-1000, 1-500, 1-400, 1-300, 1-200, 1-100, 1-50, 10-2000, 10-1000, 10-500, 10-400, 10-300, 10-200, 10-100, 10-50, 20-2000, 20-1000, 20-500, 20-400, 20-300, 20-200, 20-100, 20-50, 30-2000, 30-1000, 30-500, 30-400, 30-300, 30-200, 30-100, 30-50, 40-2000, 40-1000, 40-500, 40-400, 40-300, 40-200, 40-100, 40-50, 50-2000, 50-1000, 50-500, 50-400, 50-300, 50-200, 100-2000, 100-1000, 100-500, 100-400, 100-300, 100-200, 200-2000, 20-1000, 200-500, 200-400, 200-300, 7.5-10, 7.5-12.5, 7.5-15, 7.5-20, 7.5-30, 10-12.5, 10-15, 10-20, 10-30, 15-25, 15-30, 40-60, 45-55, 190-210, or 290-310 kDa, for example. The Mw of dextran in some additional or alternative aspects can be about, at least about, or less than about, 0.1, 0.125, 0.15, 0.175, 0.2, 0.24, 0.25, 0.5, 0.75, 1,2,3,4,5,6,7,8,9,10,20,30,40,50,60,70,80,90,100,110, 120,130, 140, 150, 160, 170, 180, 190, 200, 0.1-0.2, 0.125-0.175, 0.13-0.17, 0.135-0.165, 0.14-0.16, 0.145-0.155, 10-80, 20-70, 30-60, 40-50, 50-200, 60-200, 70-200, 80-200, 90-200, 100-200, 110-200, 120-200, 50-180, 60-180, 70-180, 80-180, 90-180, 100-180, 110-180, 120-180, 50-160, 60-160, 70-160, 80-160, 90-160, 100-160, 110-160, 120-160, 50-140, 60-140, 70-140, 80-140, 90-140, 100-140, 110-140, 120-140, 50-120, 60-120, 70-120, 80-120, 90-120, 90-110, 100-120, 110-120, 50-110, 60-110, 70-110, 80-110, 90-110, 100-110, 50-100, 60-100, 70-100, 80-100, 90-100, or 95-105 million Daltons. The molecular weight of a dextran ether herein can be calculated, for example, based on any of the foregoing dextran kDa values, further taking into account the ether's DoS and type of ether group(s); such a molecular weight can be about, at least about, or less than about, any of the above kDa values or ranges, for example. Any of the forgoing Mw values can characterize a dextran herein before, or after, it has optionally been branched (e.g., alpha-1,2 and/or alpha-1,3), for instance.

The dextran portion of a dextran ether derivative herein can be as disclosed (e.g., molecular weight, linkage/branching profile, production method), for example, in U.S. Patent Appl. Publ. Nos. 2016/0122445, 2017/0218093, 2018/0282385, 2020/0165360, or 2019/0185893, which are each incorporated herein by reference. In some aspects, a dextran for ether derivatization can be one produced in a suitable reaction comprising glucosyltransferase (GTF) 0768 (SEQ ID NO:1 or 2 of US2016/0122445), GTF 8117, GTF 6831, or GTF 5604 (these latter three GTF enzymes are SEQ ID NOs:30, 32 and 33, respectively, of US2018/0282385), or a GTF comprising an amino acid sequence that is at least 90%, 91%, 92%, 93%, 94%, 95%, 96%, 97%, 98%, or 99% identical to the amino acid sequence of GTF 0768, GTF 8117, GTF 6831, or GTF 5604.

In some aspects, an ether derivative of an alpha-glucan of the present disclosure can have a degree of substitution (DoS) up to about 3.0 (e.g., 0.001 to 3.0, or 0.01 to 3.0) with at least one positively charged (cationic) organic group that is ether-linked to the alpha-glucan. The DoS can be about, at least about, or up to about, 0.001, 0.0025, 0.005, 0.01, 0.02, 0.025, 0.03, 0.04, 0.05, 0.06, 0.07, 0.075, 0.08, 0.09, 0.1, 0.15, 0.2, 0.25, 0.3, 0.35, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 (DoS can optionally be expressed as a range between any two of these values), for example. Some examples of DoS ranges herein include 0.01-3.0, 0.01-2.5, 0.01-2.0, 0.01-1.5, 0.01-1.0, 0.01-0.5, 0.01-0.4, 0.01-0.3, 0.01-0.2, 0.01-0.175, 0.01-0.15, 0.01-0.125, 0.01-0.1, 0.05-3.0, 0.05-2.5, 0.05-2.0, 0.05-1.5, 0.05-1.0, 0.05-0.8, 0.05-0.5, 0.05-0.4, 0.05-0.3, 0.05-0.2, 0.05-0.175, 0.05-0.15, 0.05-0.125, 0.05-0.1, 0.1-3.0, 0.1-2.5, 0.1-2.0, 0.1-1.5, 0.1-1.0, 0.1-0.8, 0.1-0.6, 0.1-0.5, 0.1-0.4, 0.1-0.3, 0.1-0.2, 0.1-0.175, 0.1-0.15, 0.1-0.125, 0.2-3.0, 0.2-2.5, 0.2-2.0, 0.2-1.5, 0.2-1.0, 0.2-0.8, 0.2-0.6, 0.2-0.5, 0.2-0.4, 0.2-0.3, 0.3-3.0, 0.3-2.5, 0.3-2.0, 0.3-1.5, 0.3-1.0, 0.3-0.8, 0.3-0.6, 0.3-0.5, 0.3-0.4, 0.35-3.0, 0.35-2.5, 0.35-2.0, 0.35-1.5, 0.35-1.0, 0.35-0.8, 0.35-0.6, 0.35-0.5, 0.4-3.0, 0.4-2.5, 0.4-2.0, 0.4-1.5, 0.4-1.0, 0.4-0.8, 0.4-0.6, 0.4-0.5, 0.5-3.0, 0.5-2.5, 0.5-2.0, 0.5-1.5, 0.5-1.0, and 0.5-0.8.

Since there are at most three hydroxyl groups in a glucose monomeric unit of an alpha-glucan, the overall DoS of an alpha-glucan ether derivative can be no higher than 3.0. It would be understood by those skilled in the art that, since an alpha-glucan ether derivative as presently disclosed has a DoS with at least one type of positively charged organic group in ether linkage (e.g., between about 0.001 to about 3.0), all the positions of an alpha-glucan ether derivative cannot only be hydroxyl.

An ether derivative of an alpha-glucan of the present disclosure can be substituted with at least one positively charged organic group herein that is ether-linked to the alpha-glucan. A positively charged group can be, for example, any of those disclosed in U.S. Patent Appl. Publ. Nos. 2016/0311935, 2018/0237816, or 2020/0002646, or Int. Pat. Appl. Publ. No. WO2021/257786 (Appl. No. PCT/US2021/37756), which are incorporated herein by reference. A positively charged group can comprise a substituted ammonium group, for example. Examples of substituted ammonium groups are primary, secondary, tertiary and quaternary ammonium groups, such as can be represented by Structures I and II. An ammonium group can be substituted with alkyl group(s) and/or aryl group(s), for example. There can be one, two, or three types of alkyl and/or aryl groups in some aspects of a substituted ammonium group. An alkyl group of a substituted ammonium group herein can be a C₁-C₃₀ alkyl group, for example, such as a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, C₂₅, C₂₆, C₂₇, C₂₈, C₂₉, or C₃₀ group; each alkyl group can be the same or different in aspects with two or three alkyl substitutions. In some aspects, an alkyl group can be a C₁₀-C₁₄ alkyl group, meaning that the alkyl group can be any one of a C₁₀, C₁₁, C₁₂, C₁₃, or C₁₄ alkyl group (this particular Cn range nomenclature applies, accordingly, to other Cn ranges herein). An alkyl group can be C₁-C₂₄, C₁-C₁₈, C₄-C₂₀, C₅-C₂₀, C₆-C₂₀, C₁-C₄, C₆-C₁₈, C₈-C₁₈, C₁₀-C₁₈, C₆-C₁₆, C₈-C₁₆, C₁₀-C₁₆, C₆-C₁₄, C₈-C₁₄, C₁₀-C₁₄, C₆-C₁₂, C₈-C₁₂, or C₁₀-C₁₂ in some aspects. By disclosing a C₁₂ alkyl group, for example, it is meant that the alkyl group is twelve carbons in length and is saturated (i.e., —CH₂CH₂CH₂CH₂CH₂CH₂CH₂CH₂CH₂CH₂CH₂CH₃); this standard meaning applies, accordingly, to other alkyl groups disclosed herein. An aryl group can be a C₆, C₆-C₂₄, C₁₂-C₂₄, or C₆-C₁₈ aryl group, for example, that is optionally substituted with one or more alkyl substituents (e.g., any alkyl group disclosed herein).

A secondary ammonium alpha-glucan ether herein can comprise a monoalkylammonium group in some aspects (e.g., based on Structure I). A secondary ammonium alpha-glucan ether can be a monoalkylammonium alpha-glucan ether in some aspects, such as a monomethyl-, monoethyl-, monopropyl-, monobutyl-, monopentyl-, monohexyl-, monoheptyl-, monooctyl-, monononyl-, monodecyl-, monoundecyl-, monododecyl-, monotridecyl-, monotetradecyl-, monopentadecyl-, monohexadecyl-, monoheptadecyl-, or monooctadecyl-ammonium alpha-glucan ether. These alpha-glucan ethers can also be referred to as methyl-, ethyl-, propyl-, butyl-, pentyl-, hexyl-, heptyl-, octyl-, nonyl-, decyl-, undecyl-, dodecyl-, tridecyl-, tetradecyl-, pentadecyl-, hexadecyl-, heptadecyl-, or octadecyl-ammonium alpha-glucan ethers, respectively.

A tertiary ammonium alpha-glucan ether herein can comprise a dialkylammonium group in some aspects (e.g., based on Structure I). A tertiary ammonium alpha-glucan ether can be a dialkylammonium alpha-glucan ether in some aspects, such as a dimethyl-, diethyl-, dipropyl-, dibutyl-, dipentyl-, dihexyl-, diheptyl-, dioctyl-, dinonyl-, didecyl-, diundecyl-, didodecyl-, ditridecyl-, ditetradecyl-, dipentadecyl-, dihexadecyl-, diheptadecyl-, or dioctadecyl- ammonium alpha-glucan ether.

A quaternary ammonium alpha-glucan ether herein can comprise a trialkylammonium group in some aspects (e.g., based on Structure I). A quaternary ammonium alpha-glucan ether compound can be a trialkylammonium alpha-glucan ether in some aspects, such as trimethyl-, triethyl-, tripropyl-, tributyl-, tripentyl-, trihexyl-, triheptyl-, trioctyl-, trinonyl-, tridecyl-, triundecyl-, tridodecyl-, tritridecyl-, tritetradecyl-, tripentadecyl-, trihexadecyl-, triheptadecyl-, or trioctadecyl- ammonium alpha-glucan ether.

In some aspects, a positively charged organic group can comprise a C₄ to C₂₀ alkylene group (e.g., of any length as disclosed herein for an alkyl group). An alkylene group can comprise one, two, three, or more double-bonds, for example. An alkylene group in some aspects can comprise one or more double-bonds spanning carbons (i) 5 and 6, (ii) 6 and 7, (iii) 8 and 9, (iv) 9 and 10, (v) 11 and 12, (vi) 12 and 13, (vii) 14 and 15, and/or (viii) 15 and 16 of the alkylene group, where carbon number is counted starting from the carbon directly linked to the positively charged group (e.g., carbon-1 is linked to the nitrogen of a substituted ammonium group herein). Some combinations of double-bonds of an alkylene group include: (iv) and (vi); (iv), (vi) and (vii); and (i), (iii), (v) and (vii) (with reference to the foregoing list). While a double-bond herein of an alkylene group can be in a cis or trans orientation, it typically is in the cis orientation. An alkylene group can be derived (derivable) from a fatty acid (e.g., caproic acid, caprylic acid, capric acid, lauric acid, myristic acid, palmitic acid, stearic acid, arachidic acid, oleic acid, linoleic acid, arachidonic acid), or an acyl group (e.g., corresponding to any fatty acid herein) of a lipid (e.g., a mono-, di-, or tri-glyceride), for example.

In some aspects, a substituted ammonium group is a tertiary ammonium group in which, with reference to Structure I and/or II, R₂ is a hydrogen atom, R₃ is a methyl, ethyl, propyl, or butyl, and R₄ is any C₄ to C₂₀ alkyl or alkylene group (e.g., any herein such as a C₁₂ alkyl). In some aspects, a substituted ammonium group is a quaternary ammonium group in which, with reference to Structure I and/or II, R₂ and R₃ are each independently a methyl, ethyl, propyl, or butyl (e.g., both R₂ and R₃ are methyl, or are both ethyl), and R₄ is any C₄ to C₂₀ alkyl or alkylene group (e.g., any herein such as a C₁₂ alkyl). A tertiary or quaternary ammonium group in some aspects comprises Structure II, and has any of the foregoing R₂, R₃ and R₄ assignments. An example of a quaternary ammonium group herein comprises dodecyldimethylammonium (i.e., the ammonium nitrogen is linked to a C₁₂ alkyl group and two methyl groups).

One of the groups of a substituted ammonium group comprises one carbon, or a chain of carbons (e.g., up to 30), in ether linkage to an alpha-glucan. A carbon chain in this context can be linear, for example. Such a carbon or carbon chain can be represented by —CH₂—, —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂—, —CH₂(CH₂)₇CH₂—, —CH₂(CH₂)₈CH₂—, —CH₂(CH₂)₉CH₂—, or —CH₂(CH₂)₁₀CH₂—, for example. In some aspects, a carbon chain in this context can be branched, such as by being substituted with one or more alkyl groups (e.g., any as disclosed above such as methyl, ethyl, propyl, or butyl). The point(s) of substitution can be anywhere along the carbon chain. Examples of branched carbon chains include —CH(CH₃)CH₂—, —CH(CH₃)CH₂CH₂—, —CH₂CH(CH₃)CH₂—, —CH(CH₂CH₃)CH₂—, —CH(CH₂CH₃)CH₂CH₂—, —CH₂CH(CH₂CH₃)CH₂—, —CH(CH₂CH₂CH₃)CH₂—, —CH(CH₂CH₂CH₃)CH₂CH₂— and —CH₂CH(CH₂CH₂CH₃)CH₂—; longer branched carbon chains can also be used, if desired. In some aspects, a chain of one or more carbons (e.g., any of the above linear or branched chains) is further substituted with one or more hydroxyl groups. Examples of hydroxy- or dihydroxy (diol)-substituted chains include —CH(OH)—, —CH(OH)CH₂—, —C(OH)₂CH₂—, —CH₂CH(OH)CH₂—, —CH(OH)CH₂CH₂—, —CH(OH)CH(OH)CH₂—, —CH₂CH₂CH(OH)CH₂—, —CH₂CH(OH)CH₂CH₂—, —CH(OH)CH₂CH₂CH₂—, —CH₂CH(OH)CH(OH)CH₂—, —CH(OH)CH(OH)CH₂CH₂— and —CH(OH)CH₂CH(OH)CH₂—. In each of the foregoing examples, the first carbon atom of the chain is ether-linked to a glucose monomer of the alpha-glucan, and the last carbon atom of the chain is linked to a positively charged group (e.g., a substituted ammonium group as disclosed herein). One or more positively charged organic groups in some aspects can comprise trimethylammonium hydroxypropyl groups (Structure II, when each of R₂, R₃ and R₄ is a methyl group).

In aspects in which a carbon chain of a positively charged organic group has a substitution in addition to a substitution with a positively charged group, such additional substitution can be with one or more hydroxyl groups, oxygen atoms (thereby forming an aldehyde or ketone group), alkyl groups (e.g., methyl, ethyl, propyl, butyl), and/or additional positively charged groups, for example. A positively charged group is typically bonded to the terminal carbon atom of the carbon chain. A positively charged group can also comprise imidazoline ring-containing compounds in some aspects.

A counter ion for a positively charged organic group herein can be any suitable anion, such as an acetate, borate, bromate, bromide, carbonate, chlorate, chloride, chlorite, dihydrogen phosphate, fluoride, hydrogen carbonate, hydrogen phosphate, hydrogen sulfate, hydrogen sulfide, hydrogen sulfite, hydroxide, hypochlorite, iodate, iodide, nitrate, nitride, nitrite, oxalate, oxide, perchlorate, permanganate, phosphate, phosphide, phosphite, silicate, stannate, stannite, sulfate, sulfide, sulfite, tartrate, or thiocyanate anion.

An alpha-glucan ether in some aspects can contain one type of etherified positively charge organic group. Examples of such a positively charge organic group are as disclosed herein. Optionally, an alpha-glucan ether compound having a single type of etherified positively charge organic group can be characterized as a monoether. In some aspects, an alpha-glucan ether can contain two or more different types of etherified positively charge organic groups (i.e., a mixed ether). An alpha-glucan ether herein typically has no other types of organic groups derivatized to the alpha-glucan (e.g., a hydrophobic group that, for example, is ether- or ester-linked to the alpha-glucan).

A solvent of a composition of the present disclosure comprises water and at least about 40% (v/v or w/w) of one or more polar organic solvents, for example. In some aspects, a solvent comprises about, or at least about, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 40-90, 40-80, 40-70, 40-60, 50-90, 50-80, 50-70, 50-60, 60-90, 60-80, 60-70, 70-90, 70-80, 40-70, 40-60, 75-85, or 85-95 v/v % or w/w % of one or more polar organic solvents. The balance of a solvent typically is water only (e.g., a solvent with about 67 v/v % polar organic solvent has about 33 v/v % water), but can optionally comprise (e.g., less than 2, 1, 0.5, or 0.25 v/v %) one or more other liquids aside from a polar organic solvent. A solvent herein can optionally be characterized as an aqueous solvent given its having water. While a solvent herein typically comprises one type of polar organic solvent, two, three, or more polar organic solvents can optionally be included; in such aspects, the polar organic solvent concentration is typically that of the combination of the polar organic solvents.

A polar organic solvent in some aspects can be protic. Examples of protic polar organic solvents herein include an alcohol (e.g., methanol, ethanol, isopropanol, 1-propanol, tert-butyl alcohol, n-butanol, iso-butanol), methyl formamide and formamide. Additional examples of protic polar organic solvents herein include n-butanol, ethylene glycol, 2-methoxyethanol, 1-methoxy-2-propanol, glycerol, 1,2-propanediol, and 1,3-propanetriol.

A polar organic solvent in some aspects can be aprotic. Examples of aprotic polar organic solvents herein include acetonitrile, dimethyl sulfoxide, acetone, N,N-dimethylformamide, N,N-dimethylacetamide, tetrahydrofuran, propylene carbonate, and sulfolane. Additional examples of aprotic polar organic solvents herein include hexamethylphosphoramide, dimethylimidazolidinone (1,3-dimethyl-2-imidazolidinone), dioxane, nitromethane, and butanone. In general, ester, ketone and aldehyde solvents having no acidic hydrogen atom are other examples of aprotic polar organic solvents herein.

An alpha-glucan ether derivative can be dissolved and/or dispersed in a solvent herein of a composition of the present disclosure, for example. In some aspects, about, or at least about, 5%, 10%, 15%, 20%, 25%, 30%, 35%, 40%, 45%, 50%, 55%, 60%, 65%, 70%, 75%, 80%, 82.5%, 85%, 87.5%, 90%, 91%, 92%, 93%, 94%, 95%, 96%, 97%, 98%, 99%, 99.5%, 99.9%, or 100% by weight of an alpha-glucan ether derivative herein is dissolved in a solvent. The balance of any alpha-glucan ether derivative that remains undissolved can be dispersed in the solvent, for example.

In some aspects, about, or at least about, 5%, 10%, 15%, 20%, 25%, 30%, 35%, 40%, 45%, 50%, 55%, 60%, 65%, 70%, 75%, 80%, 82.5%, 85%, 87.5%, 90%, 91%, 92%, 93%, 94%, 95%, 96%, 97%, 98%, 99%, 99.5%, 99.9%, or 100% by weight of an alpha-glucan ether derivative herein is dispersed in a solvent. The balance of any alpha-glucan ether derivative that is not dispersed can be dissolved in the solvent, for example. A dispersion of alpha-glucan ether derivative(s) in a solvent of the disclosure can be characterized as a stable dispersion in some aspects. It is notable that a dispersion of an alpha-glucan ether derivative herein typically has enhanced stability in that the particles of the ether derivative are able to remain dispersed following formation of the dispersion. For example, in a dispersion of alpha-glucan ether particles, the particles can be dispersed through about, or at least about, 50%, 55%, 60%, 65%, 70%, 75%, 80%, 90%, 95%, 98%, 99%, 100% 60%-100%, 60%-95%, 60%-90%, 60%-85%, 60%-80%, 70%-100%, 70%-95%, 70%-90%, 70%-85%, 70%-80%, 80%-100%, 80%-95%, or 80%-90% of the volume of the dispersion. In some aspects, any of the above levels of dispersion is contemplated to be (to persist) for a time (typically beginning from initial preparation of the dispersion) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years. In some aspects, stability can additionally or alternatively refer to an alpha-glucan ether derivative herein having an enhanced ability to provide viscosity (e.g., any of the above viscosity levels disclosed herein, optionally for any of the above time periods). In some aspects, dispersion of alpha-glucan ether derivative particles in an emulsion confers stability to the emulsion; for example, any of the above dispersal-volume percentages and/or times of such stability can likewise characterize dispersed/emulsified droplets.

A composition as presently disclosed can have a turbidity of about, or less than about, 1500, 1400, 1300, 1200, 1100, 1000, 900, 800, 700, 600, 500, 400, 300, 280, 260, 240, 220, 200,190, 180, 170, 160, 150, 140, 130, 120, 110,100, 90, 80, 70, 60, 50, 45, 40, 35, 30, 25, 20, 18, 16, 14, 12, 10, 9, 8,7, 6, 5,4, 3, 2, 1,1-250, 1-200, 1-150, 1-100, 1-50, 1-20, 1-15, 1-10, 1-5, 2-250, 2-200, 2-150, 2-100, 2-50, 2-20, 2-15, 2-10, 2-5, 10-250, 10-200, 10-150, 10-100, 10-50, or 10-20 NTU (nephelometric turbidity units), for example. Any of these NTU values can optionally be with respect to the alpha-glucan ether derivative and solvent ingredients portion of a composition herein. In some aspects, any of these NTU levels is contemplated to be (to persist) for a time (typically beginning from initial preparation) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years. Any suitable method can be used to measure turbidity, such as the methodology disclosed in Progress in Filtration and Separation (Edition: 1, Chapter 16. Turbidity: Measurement of Filtrate and Supernatant Quality?, Publisher: Academic Press, Editors: E. S. Tarleton, July 2015), which is incorporated herein by reference, or as described in the below Examples.

A composition as presently disclosed can comprise about, at least about, or up to about, 0.1, 0.25, 0.5, 0.75, 1, 2, 3, 4, 5, 6, 7, 8, 9, 10, 0.1-10, 0.5-5, 0.5-4, 0.5-3, 0.5-2, 1-5, 1-4, 1-3, 1-2, 2-5, 2-4, 2-3, 0.5-1.5, 1.5-2.5, 2.5-3.5, or 3.5-4.5 wt % of one of, or a combination of (e.g., two, three, four, or more), alpha-glucan ether derivatives herein, for example. Any of these concentration values can optionally be with respect to the alpha-glucan ether derivative and solvent ingredients portion of a composition herein.

A composition as presently disclosed typically comprises at least one solvent herein and at least one alpha-glucan ether derivative herein. In some aspects, a composition comprises about, or at least about, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.25, 99.5, 99.75, or 99.9 wt % of a solvent herein.

A composition herein can have a viscosity of about, at least about, or less than about, 1, 5, 10, 100, 200, 300, 400, 500, 600, 700, 1000, 2000, 3000, 4000, 5000, 6000, 7000, 8000, 9000, 10000, 15000, 1-300, 10-300, 25-300, 50-300, 1-250, 10-250, 25-250, 50-250, 1-200, 10-200, 25-200, 50-200, 1-150, 10-150, 25-150, 50-150, 1-100, 10-100, 25-100, or 50-100 centipoise (cps), for example.

Viscosity can be as measured with a composition herein at any temperature between about 3° C. to about 80° C., for example (e.g., 4-30° C., 15-30° C., 15-25° C.). Viscosity typically is as measured at atmospheric pressure (about 760 torr) or a pressure that is ±10% thereof. Viscosity can be measured using a viscometer or rheometer, for example, and can optionally be as measured at a shear rate (rotational shear rate) of about 0.1, 0.5, 1.0, 5, 10, 50, 100, 500, 1000, 0.1-500, 0.1-100, 1.0-500, 1.0-1000, or 1.0-100 s⁻¹ (1/s), or about 5, 10, 20, 25, 50, 100, 200, or 250 rpm (revolutions per minute), for example.

A composition in some aspects has no (detectable) dissolved sugars, or about 0.1-1.5, 0.1-1.25, 0.1-1.0, 0.1-0.75, 0.1-0.5, 0.2-0.6, 0.3-0.5, 0.2, 0.3, 0.4, 0.5, or 0.6 wt % dissolved sugars. Such dissolved sugars can include sucrose, fructose, leucrose, and/or soluble gluco-oligosaccharides, for example. A composition in some aspects can have one or more salts/buffers (e.g., Na⁺, Cl⁻, NaCl, phosphate, tris, citrate) (e.g., ≤0.1, 0.5, 1.0, 2.0, or 3.0 wt %), and/or a pH of about 4.0, 4.5, 5.0, 5.5, 6.0, 6.5, 7.0, 7.5, 8.0, 8.5, 9.0, 9.5, 10.0, 10.5, 4.0-10.0, 4.0-9.0, 4.0-8.0, 5.0-10.0, 5.0-9.0, 5.0-8.0, 6.0-10.0, 6.0-9.0, or 6.0-8.0, for example.

The temperature of a composition herein can be about, at least about, or up to about, 0, 5, 10, 15, 20, 25, 30, 35, 40, 45, 50, 55, 60, 65, 70, 75, 80, 85, 90, 95, 100, 105, 110, 115, 120, 125, 130, 5-50,20-25, 20-30, 20-35, 20-40, 30-40, 30-45, 30-50, 40-130, 40-125, 40-120, 70-130, 70-125, 70-120, 80-130, 80-125, 80-120, 60-100, 60-90, 70-100, 70-90, 75-100, 75-90, or 75-85° C., for example.

A composition herein can be a detergent composition in some aspects. Examples of such compositions are disclosed herein as detergents for dishwashing and detergents for fabric care.

A composition herein can, in some aspects, comprise one or more salts such as a sodium salt (e.g., NaCl, Na₂SO₄). Other non-limiting examples of salts include those having (i) an aluminum, ammonium, barium, calcium, chromium (II or III), copper (I or II), iron (II or Ill), hydrogen, lead (II), lithium, magnesium, manganese (II or Ill), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous composition herein at a wt % of about, or at least about, 0.01, 0.025, 0.05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

A composition herein can optionally contain one or more enzymes (active enzymes). Examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases, nucleases (e.g., deoxyribonuclease or ribonuclease), and amylases. If an enzyme(s) is included, it may be comprised in a composition herein at about 0.0001-0.1 wt % (e.g., 0.01-0.03 wt %) active enzyme (e.g., calculated as pure enzyme protein), for example. In fabric care or automatic dishwashing applications, an enzyme herein (e.g., any of the above such as cellulase, protease, amylase, and/or lipase) can be present in an aqueous composition in which a fabric or dish is treated (e.g., wash liquor, grey water) at a concentration that is minimally about 0.01-0.1 ppm total enzyme protein, or about 0.1-10 ppb total enzyme protein (e.g., less than 1 ppm), to maximally about 100, 200, 500, 1000, 2000, 3000, 4000, or 5000 ppm total enzyme protein, for example.

An alpha-glucan ether derivative and/or a composition comprising such a derivative is biodegradable in some aspects. Such biodegradability can be, for example, as determined by the Carbon Dioxide Evolution Test Method (OECD Guideline 301B, incorporated herein by reference), to be about, at least about, or at most about, 5%, 10%, 15%, 20%, 25%, 30%, 35%, 40%, 45%, 50%, 55%, 60%, 65%, 70%, 75%, 80%, 85%, 90%, 5-60%, 5-80%, 5-90%, 40-70%, 50-70%, 60-70%, 40-75%, 50-75%, 60-75%, 70-75%, 40-80%, 50-80%, 60-80%, 70-80%, 40-85%, 50-85%, 60-85%, 70-85%, 40-90%, 50-90%, 60-90%, or 70-90%, or any value between 5% and 90%, after 15, 30, 45, 60, 75, or 90 days of testing.

A composition can comprise one, two, three, four or more different alpha-glucan ether derivatives herein and, optionally, at least one non-derivatized alpha-glucan (e.g., as disclosed herein). For example, a composition can comprise at least one type of alpha-glucan ether derivative and at least one type of alpha-glucan; in some aspects, the latter can be a precursor compound of the former. In some aspects, a non-derivatized alpha-glucan (e.g., precursor compound) is not present.

A composition herein comprising at least a solvent and an ether derivative of an alpha-glucan can be as produced by a method (as described below) for producing such a composition, for example.

Some aspects of the present disclosure concern a method of producing a composition herein. Such a method can comprise mixing together a solvent and one or more alpha-glucan ether derivatives, thereby producing the composition. Such a method can optionally be characterized as a mixing or blending method/process.

A solvent in a mixing method herein can be as presently disclosed, for example. A solvent can have any relevant feature of a composition herein such as temperature, pH, salt/buffer content, water content, and/or polar organic solvent content, for example. An alpha-glucan ether derivative in a mixing method herein can be as presently disclosed, for example. A composition produced by a mixing method herein can have any relevant feature herein such as temperature, pH, salt/buffer content, alpha-glucan ether solubility and/or dispersibility (e.g., stable dispersibility), and/or turbidity, for example.

Mixing of at least a solvent and an alpha-glucan ether derivative in a mixing method herein can entail one or more of stirring, shaking, vortexing, agitation, blending, paddling, rotating, sonication, comminuting, and/or shearing, for example. In some aspects, blending can be performed by, or further include, using a sonicator (e.g., ultrasonicator) (e.g., 40-60 W, ˜50 W), homomixer, high shear mixer or homogenizer (e.g., rotary or piston, rotor-stator [in-line rotor-stator], Waring®blender), planetary mixer, colloid mill, jet mill, vortex, and/or any other suitable methodology.

In some aspects, one or more alpha-glucan ether derivatives as presently disclosed can first be dissolved in water or an aqueous solution or mixture having about, or less than about, 20%, 15%, 10%, 5%, or 2.5% (v/v or w/w) of a polar organic solvent herein to form a first preparation. A first preparation can optionally contain, before or after dissolving alpha-glucan ether(s) therein, one or more ingredients such as any of a household care product, personal care product, industrial product, ingestible product (e.g., food product), or pharmaceutical product herein. A polar organic solvent (e.g., the same as that which is optionally already present in the first preparation) can then be blended into the first preparation to a concentration as disclosed herein. Optionally, one or more other ingredients can then be added to this preparation. In some aspects, addition of polar organic solvent to a first preparation can be conducted over a period of about, or at least about, 1, 2, 3, 4, 5, 6, 7, 8, 1-6, 1-5, 1-4, 2-6, 2-5, 2-4, 3-6, 3-5, 3-4, 4-6, or 4-5 hours. Addition of a polar organic solvent for this or another time period can be done by any suitable means such as portion-wise, dropwise, continuous stream/flow, or other uniform addition method. A portion-wise addition approach can comprise, for example, adding each portion of the entire scheduled dose of polar organic solvent in uniformly divided time increments, where each addition typically is followed by mixing (e.g., as above). An “entire scheduled dose” is the volume of polar organic solvent needed to render a desired concentration of the polar organic solvent in the solvent of a composition herein. A portion can be ¼th, ⅕th, ⅙th, ⅛th, 1/10th, 1/12th, 1/14^{th}, 1/15th, 1/16th, 1/18th, or 1/20^{th} of the volume of the entire scheduled dose, and the time increments for such addition can be calculated by dividing the total scheduled addition time by the number of portions to be added, but taking into account that addition of the first dose of polar organic solvent is typically taken at timepoint zero (time 0).

A composition as presently disclosed comprising at least a solvent and an alpha-glucan ether derivative can be in the form of, or comprise, a household care product, personal care product, industrial product, ingestible product (e.g., food product), medical product, or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or Int. Patent Appl. Publ. No. WO2016/133734, which are all incorporated herein by reference. In some aspects, a composition can comprise at least one component/ingredient of a household care product, personal care product, industrial product, pharmaceutical product, or ingestible product (e.g., food product) as disclosed in any of the foregoing publications and/or as presently disclosed.

Personal care products herein are not particularly limited and include, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended pharmacological effect.

A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, and orange oil. A skin care product can be an ointment, lotion, or sanitizer (e.g., hand sanitizer) in some aspects.

A personal care product herein can also be in the form of makeup, lipstick, mascara, rouge, foundation, blush, eyeliner, lip liner, lip gloss, other cosmetics, sunscreen, sun block, nail polish, nail conditioner, temporary tattoo ink, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse (e.g., hair styling mousse), hair spray (e.g., hair styling spray), and styling gel (e.g., hair styling gel). A hair care product can be in the form of a liquid, paste, gel, solid, or powder in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine; hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

A composition in some aspects can be a hair care composition such as a hair styling or hair setting composition (e.g., hair spray, hair gel or lotion, hair mousse/foam) (e.g., aerosol hair spray, non-aerosol pump-spray, spritze, foam, crème, paste, non-runny gel, mousse, pomade, lacquer, hair wax). A hair styling/setting composition/formulation that can be adapted to a composition herein can be as disclosed in, for example, US20090074697, WO1999048462, US20130068849, JPH0454116A, U.S. Pat. No. 5,304,368, AU667246B2, U.S. Pat. Nos. 5,413,775, 5,441,728, 5,939,058, JP2001302458A, U.S. Pat. No. 6,346,234, US20020085988, U.S. Pat. No. 7,169,380, US20090060858, US20090326151, US20160008257, WO2020164769, or US20110217256, all of which are incorporated herein by reference. A hair care composition such as a hair styling/setting composition can comprise one or more ingredients/additives as disclosed in any of the foregoing references, and/or one or more of a fragrance/perfume, aroma therapy essence, herb, infusion, antimicrobial, stimulant (e.g., caffeine), essential oil, hair coloring, dying or tinting agent, anti-gray agent, anti-foam agent, sunscreen/UV-blocker (e.g., benzophenone-4), vitamin, antioxidant, surfactant or other wetting agent, mica, silica, metal flakes or other glitter-effect material, conditioning agent (e.g., a volatile or non-volatile silicone fluid), anti-static agent, opacifier, detackifying agent, penetrant, preservative (e.g., phenoxyethanol, ethylhexylglycerin, benzoate, diazolidinyl urea, iodopropynyl butylcarbamate), emollient (e.g., panthenol, isopropyl myristate), rheology-modifying or thickening polymer (e.g., acrylates/methacrylamide copolymer, polyacrylic acid [e.g., CARBOMER]), emulsified oil phase, petrolatum, fatty alcohols, diols and polyols, emulsifier (e.g., PEG-40 hydrogenated castor oil, Oleth-20), humectant (e.g., glycerin, caprylyl glycol), silicone derivative, protein, amino acid (e.g., isoleucine), conditioner, chelant (e.g., EDTA), solvent (e.g., see below), monosaccharide (e.g., dextrose), disaccharide, oligosaccharide, pH-stabilizing compound (e.g., aminomethyl propanol), film former (e.g., acrylates/hydroxyester acrylate copolymer, polyvinylpyrrolidone/vinyl acetate copolymer, triethyl acetate), aerosol propellant (e.g., C₃-C₅ alkane such as propane, isobutane, or n-butane, monoalkyl ether, dialkyl ether such as di(C₁-C₄ alkyl) ether [e.g., dimethyl ether]), and/or any other suitable material herein. An alpha-glucan ether derivative as used in a hair styling/setting composition herein can function as a hair fixing/styling agent (typically non-permanent hair fixing, but durable), for example, and optionally is the only hair fixing agent in the composition. Optional additional hair fixing/styling agents herein include PVP (polyvinylpyrrolidone), octylacrylamide/acrylates/butylaminoethyl methacrylate copolymer, vinyl caprolactam/PVP/dimethylaminoethyl methacrylate copolymer, AMPHOMER, or any film former such as listed above.

The total content of one or more alpha-glucan ether derivatives in a hair care composition such as a hair styling/setting composition herein can be about, at least about, or less than about, 0.5, 1, 2, 3, 4, 5, 6, 7, 8, 9, 10, 11, 12, 13, 14, 15, 0.5-15, 0.5-10, 0.5-5, 0.5-2, 1-15, 1-10, 1-5, 1-2, 2.5-7.5, 3-7, or 4-6 wt %, for example. A hair styling/setting composition can comprise a solvent comprising water and a water-miscible (typically polar) organic solvent such as an alcohol (e.g., ethanol, propanol, isopropanol, n-butanol, iso-butanol, tert-butanol), an alkylene glycol alkyl ether, and/or a monoalkyl or dialkyl ether (e.g., dimethyl ether), or any other polar organic solvent herein, for example. The amount of a polar organic solvent in a hair styling/setting composition herein can be about 50-90, 60-90, 70-90, 80-90, 50-95, 60-95, 70-95, 80-95, or 90-95 wt % or % v/v, for example.

An example of a hair styling gel formulation herein can comprise about 90-95 wt % (e.g., ˜92 wt %) solvent (e.g., solvent herein comprising water and polar organic solvent), 0.3-1.0 wt % (e.g., ˜0.5 wt %) thickener (e.g., polyacrylic acid), 0.1-0.3 wt % (e.g., ˜0.2 wt %) chelant (e.g., EDTA) (optional), 0.2-1.0 wt % (e.g., ˜0.5 wt %) humectant (e.g., glycerin), 0.01-0.05 wt % (e.g., ˜0.02 wt %) UV-blocker (e.g., benzophenone-4) (optional), 0.05-0.3 wt % (e.g., ˜0.1 wt %) preservative (e.g., diazolidinyl urea) (optional), 0.5-1.2 wt % (e.g., ˜0.8 wt %) emulsifier (e.g., Oleth-20), 0.1-0.3 wt % (e.g., ˜0.2 wt %) fragrance/perfume (optional), 0.2-1.0 wt % (e.g., ˜0.5 wt %) pH-stabilizing compound (e.g., aminomethyl propanol), and 3-7 wt % (e.g., ˜5 wt %) alpha-glucan ether derivative herein (e.g., as a hair fixing/styling agent).

An example of a hair styling spray formulation herein can comprise about 0.2-1.0 wt % (e.g., ˜0.5 wt %) pH-stabilizing compound (e.g., aminomethyl propanol), 0.1-0.3 wt % (e.g., ˜0.2 wt %) fragrance/perfume (optional), 0.05-0.12 wt % (e.g., ˜0.08 wt %) surfactant (e.g., ethoxylated dimethicone polyol), 0.05-0.12 wt % (e.g., ˜0.08 wt %) conditioner (e.g., cyclomethicone) (optional), 0.05-0.3 wt % (e.g., ˜0.2 wt %) preservative (e.g., sodium benzoate) (optional), 15-20 wt % (e.g., ˜17 wt %) water, 30-40 wt % (e.g., ˜65 wt %) alcohol (e.g., ethanol), 40-60 wt % (e.g., ˜45 wt %) propellant (e.g., dimethyl ether, or a ˜2:1 mix of dimethyl ether to C₃-C₅ alkane [e.g., mix of propane and isobutane]), and 2-4 wt % (e.g., ˜2.75 wt %) alpha-glucan ether derivative herein (e.g., as a hair fixing/styling agent).

Some aspects of the present disclosure regard hair that has been treated with a hair care composition herein (e.g., hair styling/setting composition, shampoo, or conditioner). For example, hair can comprise an alpha-glucan ether derivative on its surface, such as in a film/coating of the hair, and/or adsorbed or otherwise deposited on the hair surface; optionally, one or more other ingredients of a hair care composition herein can also be present. Typically, hair as presently disclosed, such as hair with a coating comprising an alpha-glucan ether, does not exhibit flaking to the naked eye (i.e., little or no noticeable flaking).

A pharmaceutical product herein can be in the form of an emulsion, liquid, elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts. A composition herein can be used as an excipient for medicaments and drugs, for example.

A household and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; polymer emulsions; latex; gels such as water-based gels; surfactant solutions; paints such as water-based paints; protective coatings; adhesives; sealants and caulks; inks such as water-based ink; metal-working fluids; films or coatings; or emulsion-based metal cleaning fluids used in electroplating, phosphatizing, galvanizing and/or general metal cleaning operations, for example. In some aspects, a composition herein is comprised in a fluid as a viscosity modifier and/or friction reducer; such uses include downhole operations/fluids (e.g., in hydraulic fracturing and enhanced oil recovery), for example.

In some aspects, a composition as presently disclosed comprising at least a solvent and an alpha-glucan ether derivative can be in the form of, or comprise, a fabric care composition. A fabric care composition can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition. In other embodiments, a fabric care composition can be in a dry form such as a granular detergent or dryer-added fabric softener sheet. Other non-limiting examples of fabric care compositions can include: liquid, gel or paste-form all-purpose or heavy-duty washing agents; liquid fine-fabric (e.g. delicates) detergents; cleaning auxiliaries such as bleach additives, “stain-stick”, or pre-treatments; substrate-laden products such as wetted wipes, pads, or sponges; sprays and mists; unit dose articles. As further examples, a composition herein can be in the form of a liquid, a gel, a hydrocolloid, an aqueous solution, a single compartment sachet, a multi-compartment sachet, a single compartment pouch, or a multi-compartment pouch.

A detergent composition herein may be in any useful form, e.g., as pastes, unit dose, or liquid. A liquid detergent in some aspects can be in the form of a compact gel containing only about 30 wt % water.

A detergent composition (e.g., of a fabric care product or any other product herein) typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein can optionally comprise one or more detergent builders or builder systems. In some aspects, oxidized alpha-1,3-glucan can be included as a co-builder; oxidized alpha-1,3-glucan compounds for use herein are disclosed in U.S. Patent Appl. Publ. No. 2015/0259439. In some aspects incorporating at least one builder, the cleaning compositions comprise at least about 1%, from about 3% to about 60%, or even from about 5% to about 40%, builder by weight of the composition. Examples of builders include alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949).

In some embodiments, suitable builders can include phosphate builders and non-phosphate builders. In some embodiments, a builder is a phosphate builder. In some embodiments, a builder is a non-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer-inhibiting agents. Suitable polymeric dye transfer-inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer-inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetraacetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo- or co-polymeric acids or their salts, in which the polycarboxylic acid comprises at least two carboxyl radicals separated from each other by not more than two carbon atoms.

A detergent composition herein may additionally comprise one or more enzymes as disclosed above, for example. In some aspects, a detergent composition can comprise one or more enzymes, each at a level from about 0.00001% to about 10% by weight of the composition and the balance of cleaning adjunct materials by weight of composition. In some other aspects, a detergent composition can also comprise each enzyme at a level of about 0.0001% to about 10%, about 0.001% to about 5%, about 0.001% to about 2%, or about 0.005% to about 0.5%, by weight of the composition. Enzymes comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in some aspects may comprise one or more other types of polymer in addition to an alpha-glucan ether derivative as disclosed herein. Examples of other types of polymers useful herein include carboxymethyl cellulose (CMC), dextran, poly(vinylpyrrolidone) (PVP), polyethylene glycol (PEG), poly(vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H₂O₂ source such as perborate or percarbonate, which may be combined with a peracid-forming bleach activator such as tetraacetylethylenediamine (TAED) or nonanoyloxybenzenesulfonate (NOBS). Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

Examples of suitable anti-redeposition and/or clay soil removal agents for a fabric care product herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and International Patent Appl. Publ. No. WO95/32272, all of which are incorporated herein by reference.

Particular forms of detergent compositions that can be adapted for purposes herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911 A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (Hlc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids (examples for which include polysaccharides, cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacrylamides, and mixtures thereof.

A detergent herein such as a heavy duty laundry detergent composition may optionally further include at least one dye transfer-inhibiting agent, examples of which are described above.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). A structurant can also be referred to as a structural agent.

A detergent herein such as that for fabric care (e.g., laundry) can be comprised in a unit dose (e.g., sachet or pouch), for example. A unit dose form can comprise a water-soluble outer film that completely envelopes a liquid detergent composition. A unit dose can comprise a single compartment, or at least two, three, or more (multiple) compartments. Multiple compartments can be arranged in a superposed orientation or a side-by-side orientation. A unit dose herein is typically a closed structure of any form/shape suitable for holding and protecting its contents without allowing contents release prior to contact with water.

Compositions disclosed herein comprising at least a solvent and an alpha-glucan ether derivative can be in the form of, or comprise, a fabric softener (liquid fabric softener), for example. An example of such a composition is a rinse used in laundering a fabric-comprising material herein typically following cleaning of the fabric-comprising material with a laundry detergent composition (e.g., laundry rinse such as used in a laundry rinse cycle in a washing machine). The concentration of an alpha-glucan ether in a composition comprising fabric softener (e.g., a rinse) can be about, or at least about, 20, 30, 40, 50, 60, 70, 80, 20-80, 20-70, 20-60, 30-80, 30-70, 30-60, 40-80, 40-70, or 40-60 ppm, for example. The concentration of a fabric softener in a composition (e.g., a rinse) can be about, or at least about, 50, 75, 100, 150, 200, 300, 400, 500, 600, 50-600, 50-500, 50-400, 50-300, 50-200, 100-600, 100-500, 100-400, 100-300, 100-200, 10-600, 50-500, 50-400, 50-300, 50-200, 200-600, 200-500, 200-400, or 200-300 ppm, for example. Fabric softener concentration can be based on the total fabric softener composition added (not necessarily based on an individual component of the fabric softener), or based on one or more fabric softening agents(s) in the fabric softener formulation. A fabric softener herein can further comprise, for example, one or more of a fabric softening agent (e.g., diethyl ester dimethyl ammonium chloride), anti-static agent, perfume, wetting agent, viscosity modifier (e.g., calcium chloride), pH buffer/buffering agent (e.g., formic acid), antimicrobial agent, anti-oxidant, radical scavenger (e.g., ammonium chloride), chelant/builder (e.g., diethylenetriamine pentaacetate), anti-foaming agent/lubricant (e.g., polydimethylsiloxane), preservative (e.g., benzisothiazolinone) and colorant. In some aspects, a fabric softener can further comprise one or more of a fabric softening agent, viscosity modifier, pH buffer/buffering agent, radical scavenger, chelant/builder and anti-foaming agent/lubricant. A fabric softener can be perfume-free and/or dye-free, or have less than about 0.1 wt % of a perfume and/or dye in some aspects. In some aspects, a fabric softener that can be adapted for use herein can be as disclosed in any of U.S. Patent Appl. Publ. Nos. 2014/0366282, 2001/0018410, 2006/0058214, 2021/0317384, or 2006/0014655, or Int. Patent Appl. Publ. Nos. WO2007/078782, WO1998/016538, WO1998/012293, WO1998007920, WO2000/070004, WO2009/146981, WO2000/70005, or WO2013087366, which are each incorporated herein by reference. Some brands of fabric softeners that can be adapted for use herein, if desired, include DOWNY, DOWNY ULTRA, DOWNY INFUSIONS, ALL, SNUGGLE, LENOR and GAIN. A liquid fabric softener product (e.g., as it exists before being used in a laundry rinse cycle) can be formulated to include at least a solvent and an alpha-glucan ether derivative in some aspects. A fabric softener in some aspects can be in a unit dose, such as disclosed herein for a detergent.

Compositions disclosed herein comprising at least a solvent and an alpha-glucan ether derivative can be in the form of, or comprise, a dishwashing detergent composition, for example. Examples of dishwashing detergents include automatic dishwashing detergents (typically used in dishwasher machines) and hand-washing dish detergents. A dishwashing detergent composition can be in any liquid/aqueous form as disclosed herein, for example. Components that may be included in some aspects of a dishwashing detergent composition include, for example, one or more of a phosphate; oxygen- or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly(oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N,N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl- and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt(III) and related complexes); (vii) a metal care agent in the range from about 0.1 wt % to 5 wt % (e.g., benzatriazoles, metal salts and complexes, and/or silicates); (viii) a glass corrosion inhibitor in the range of about 0.1 wt % to 5 wt % (e.g., a salt and/or complex of magnesium, zinc, or bismuth); and/or (ix) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts). In some aspects, a dishwashing detergent ingredient or entire composition (but adapted accordingly to comprise an alpha-glucan ether derivative herein) can be as disclosed in U.S. Pat. No. 8,575,083 or 9,796,951, or U.S. Pat. Appl. Publ. No. 2017/0044468, which are each incorporated herein by reference.

A detergent herein such as that for dish care can be comprised in a unit dose (e.g., sachet or pouch) (e.g., water-soluble unit dose article), for example, and can be as described above for a fabric care detergent, but rather comprise a suitable dish detergent composition.

Compositions disclosed herein comprising at least a solvent and an alpha-glucan ether derivative can be in the form of, or comprise, an oral care composition, for example. Examples of oral care compositions include dentifrices, toothpaste, mouth wash, mouth rinse, and chewing gum that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ˜0.1-10 wt % or ˜0.1-5.0 wt %, ˜0.1-2.0 wt %) of an alpha-glucan ether derivative as disclosed herein, for example. An alpha-glucan ether derivative comprised in an oral care composition can sometimes be provided therein as a thickening agent and/or dispersion agent, which may be useful to impart a desired consistency and/or mouth feel to the composition. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which an alpha-glucan ether derivative herein can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris(2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g.,azacycloalkane-2,2-diphosphonates such as azacycloheptane-2,2-diphosphonic acid), N-methyl azacyclopentane-2,3-diphosphonic acid, ethane-1-hydroxy-1,1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri- and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean- and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition.

An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors.

Additional examples of personal care, household care, and other products and ingredients herein can be any as disclosed in U.S. Pat. No. 8,796,196, which is incorporated herein by reference. Examples of personal care, household care, and other products and ingredients herein include perfumes, fragrances, air odor-reducing agents, insect repellents and insecticides, bubble-generating agents such as surfactants, pet deodorizers, pet insecticides, pet shampoos, disinfecting agents, hard surface (e.g., floor, tub/shower, sink, toilet bowl, door handle/panel, glass/window, car/automobile exterior or interior) treatment agents (e.g., cleaning, disinfecting, and/or coating agents), wipes and other non-woven materials, colorants, preservatives, antioxidants, emulsifiers, emollients, oils, medicaments, flavors, and suspending agents.

The present disclosure also concerns a method of treating a material. This method comprises contacting a material with a composition herein comprising at least a solvent and an alpha-glucan ether derivative.

A material contacted with a composition in a contacting method herein can comprise a fabric in some aspects. A fabric herein can comprise natural fibers, synthetic fibers, semi-synthetic fibers, or any combination thereof. A semi-synthetic fiber herein is produced using naturally occurring material that has been chemically derivatized, an example of which is rayon. Non-limiting examples of fabric types herein include fabrics made of (i) cellulosic fibers such as cotton (e.g., broadcloth, canvas, chambray, chenille, chintz, corduroy, cretonne, damask, denim, flannel, gingham, jacquard, knit, matelassé, oxford, percale, poplin, plisse, sateen, seersucker, sheers, terry cloth, twill, velvet), rayon (e.g., viscose, modal, lyocell), linen, and Tencel®; (ii) proteinaceous fibers such as silk, wool and related mammalian fibers; (iii) synthetic fibers such as polyester, acrylic, nylon, and the like; (iv) long vegetable fibers from jute, flax, ramie, coir, kapok, sisal, henequen, abaca, hemp and sunn; and (v) any combination of a fabric of (i)-(iv). Fabric comprising a combination of fiber types (e.g., natural and synthetic) include those with both a cotton fiber and polyester, for example. Materials/articles containing one or more fabrics herein include, for example, clothing, curtains, drapes, upholstery, carpeting, bed linens, bath linens, tablecloths, sleeping bags, tents, car interiors, etc. Other materials comprising natural and/or synthetic fibers include, for example, non-woven fabrics, paddings, paper, and foams.

A composition that is contacted with a fabric can be, for example, a fabric care composition (e.g., laundry detergent, fabric softener). Thus, a treatment method in certain embodiments can be considered a fabric care method or laundry method if employing a fabric care composition therein. A fabric care composition herein is contemplated to effect one or more of the following fabric care benefits (i.e., surface substantive effects): wrinkle removal, wrinkle reduction, wrinkle resistance, fabric wear reduction, fabric wear resistance, fabric pilling reduction, extended fabric life, fabric color maintenance, fabric color fading reduction, reduced dye transfer, fabric color restoration, fabric soiling reduction, fabric soil release, fabric shape retention, fabric smoothness enhancement, anti-redeposition of soil on fabric, anti-greying of laundry, improved fabric hand/handle, and/or fabric shrinkage reduction.

Examples of conditions (e.g., time, temperature, wash/rinse volumes) for conducting a fabric care method or laundry method herein are disclosed in WO1997/003161 and U.S. Pat. Nos. 4,794,661, 4,580,421 and 5,945,394, which are incorporated herein by reference. In other examples, a material comprising fabric can be contacted with an aqueous composition herein: (i) for at least about 5, 10, 20, 30, 40, 50, 60, 70, 80, 90, 100, 110, or 120 minutes; (ii) at a temperature of at least about 10, 15, 20, 25, 30, 35, 40, 45, 50, 55, 60, 65, 70, 75, 80, 85, 90, or 95° C. (e.g., for laundry wash or rinse: a “cold” temperature of about 15-30° C., a “warm” temperature of about 30-50° C., a “hot” temperature of about 50-95° C.); (iii) at a pH of about 2, 3, 4, 5, 6, 7, 8, 9, 10, 11, or 12 (e.g., pH range of about 2-12, or about 3-11); (iv) at a salt (e.g., NaCl) concentration of at least about 0.5, 1.0, 1.5, 2.0, 2.5, 3.0, 3.5, or 4.0 wt %; or any combination of (i)-(iv).

The contacting step in a fabric care method or laundry method can comprise any of washing, soaking, and/or rinsing steps, for example. Contacting a material or fabric in still further embodiments can be performed by any means known in the art, such as dissolving, mixing, shaking, spraying, treating, immersing, flushing, pouring on or in, combining, painting, coating, applying, affixing to, and/or communicating an effective amount of an alpha-glucan ether derivative herein with the fabric or material. In still further embodiments, contacting may be used to treat a fabric to provide a surface substantive effect. As used herein, the term “fabric hand” or “handle” refers to a person's tactile sensory response towards fabric which may be physical, physiological, psychological, social or any combination thereof. In one embodiment, the fabric hand may be measured using a PhabrOmeter® System for measuring relative hand value (available from Nu Cybertek, Inc. Davis, CA) (American Association of Textile Chemists and Colorists [AATCC test method “202-2012, Relative Hand Value of Textiles: Instrumental Method” ]).

In some aspects of treating a material comprising fabric, an alpha-glucan ether derivative of the composition adsorbs to the fabric. This feature is believed to render an alpha-glucan ether derivative herein useful as an anti-redeposition agent and/or anti-greying agent in fabric care compositions (in addition to its viscosity-modifying effect, e.g.). An anti-redeposition agent or anti-greying agent herein helps keep soil from redepositing onto clothing in wash water after the soil has been removed. It is further contemplated that adsorption of an alpha-glucan ether herein to a fabric enhances mechanical properties of the fabric in some aspects.

Adsorption of an alpha-glucan ether derivative to a fabric herein can be measured using a colorimetric technique (e.g., Dubois et al., 1956, Anal. Chem. 28:350-356; Zemljič et al., 2006, Lenzinger Berichte 85:68-76; both incorporated herein by reference), for example, or any other method known in the art.

Other materials that can be contacted in the above treatment method include surfaces that can be treated with a dish detergent (e.g., automatic dishwashing detergent or hand dish detergent). Examples of such materials include surfaces of dishes, glasses, pots, pans, baking dishes, utensils and flatware made from ceramic material, china, metal, glass, plastic (e.g., polyethylene, polypropylene, polystyrene, melamine, etc.) and wood (collectively referred to herein as “tableware”). Thus, the treatment method in certain embodiments can be considered a dishwashing method or tableware washing method, for example. Examples of conditions (e.g., time, temperature, wash volume) for conducting a dishwashing or tableware washing method herein are disclosed herein and in U.S. Pat. No. 8,575,083 and U.S. Pat. Appl. Publ. No. 2017/0044468, which are incorporated herein by reference. In some aspects, a tableware article can be contacted with a composition herein under a suitable set of conditions such as any of those disclosed above with regard to contacting a fabric-comprising material.

Other materials that can be contacted in the above treatment method include oral surfaces such as any soft or hard surface within the oral cavity including surfaces of the tongue, hard and soft palate, buccal mucosa, gums and dental surfaces (e.g., natural tooth or a hard surface of artificial dentition such as a crown, cap, filling, bridge, denture, or dental implant). Thus, a treatment method in certain embodiments can be considered an oral care method or dental care method, for example. Conditions (e.g., time, temperature) for contacting an oral surface with an aqueous composition herein should be suitable for the intended purpose of making such contact. Other surfaces that can be contacted in a treatment method herein include a surface of the integumentary system such as skin, hair, or nails.

Thus, some aspects of the present disclosure concern material (e.g., fabric, or a fiber-comprising product as disclosed herein, or any other material herein such as hair or skin) that comprises an alpha-glucan ether derivative herein. Such material can be produced following a material treatment method as disclosed herein, for example. A material may comprise an alpha-glucan ether derivative in some aspects if the alpha-glucan ether derivative is adsorbed to, or otherwise in contact with, the surface of the material.

Some aspects of a method of treating a material herein further comprise a drying step, in which a material is dried after being contacted with the composition. A drying step can be performed directly after the contacting step, or following one or more additional steps that might follow the contacting step (e.g., drying of fabric or tableware after being rinsed, in water for example, following a wash in a composition herein). Drying can be performed by any of several means known in the art, such as air drying (e.g., ˜20-25° C.), or at a temperature of at least about 30, 40, 50, 60, 70, 80, 90, 100, 120, 140, 160, 170, 175, 180, or 200° C., for example. A material that has been dried herein typically has less than 3, 2, 1, 0.5, or 0.1 wt % water comprised therein.

An aqueous composition used in a treatment method herein can be any aqueous composition disclosed herein. Examples of aqueous compositions include detergents (e.g., laundry detergent or dish detergent), fabric softeners, water-containing dentifrices such as toothpaste, and hair care products such as hair styling, hair cleaning, or hair conditioning products.

Thus, the present disclosure also concerns a method of producing a film or coating. Such a method can comprise: (a) providing a composition herein comprising at least a solvent and an alpha-glucan ether derivative, (b) contacting the composition with a surface, and (c) removing at least about 95% by weight of the solvent to form a film or coating on the surface. A surface can be that of any material disclosed herein, for example. A film or coating as produced by such a method can be as disclosed herein, for example.

A film or coating herein can be a dried film or coating in some aspects, comprising less than about 5, 4, 3, 2, 1, 0.5, or 0.1 wt % water, for example. The amount of alpha-glucan ether derivative comprised in a film or coating herein can be about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, or 99.9 wt %, for example.

A film or coating herein can have a thickness of about, or at least about, 0.5, 0.6, 0.7, 0.8, 0.9, 1.0, 1.1, 1.2, 1.3, 1.4, 1.5, 0.5-1.5, 0.8-1.5, 1.0-1.5, 0.5-1.4, 0.8-1.4, or 1.0-1.4 mil, for instance (1 mil=0.001 inch). In some aspects, such thickness is uniform, which can be characterized by having a contiguous area that (i) is at least 20%, 30%, 40%, or 50% of the total film/coating area, and (ii) has a standard deviation of thickness of less than about 0.06, 0.05, or 0.04 mil.

A film or coating herein can exhibit various degrees of transparency as desired. For example, a film/coating can be highly transparent (e.g., high optical transparency, and/or low haze). Optical transparency as used herein can refer to a film or coating allowing at least about 10-99% light transmission, or at least about 50%, 60%, 70%, 80%, 90%, 95%, 96%, 97%, 98%, or 99% light transparency, for example. High transparency can optionally refer to a film/coating having at least about 90% optical transmittance. Transparency of a film/coating herein can be measured following test ASTM D 1746 (2009, Standard Test Method for Transparency of Plastic Sheeting, ASTM International, West Conshohocken, PA), for example, which is incorporated herein by reference.

A film or coating herein can optionally further comprise a plasticizer such as glycerol, propylene glycol, ethylene glycol, and/or polyethylene glycol. In some aspects, other film components (in addition to an alpha-glucan ether derivative herein) can be as disclosed in U.S. Patent. Appl. Publ. No. 2011/0151224 or 2015/0191550, or U.S. Pat. No. 9,688,035 or 3,345,200, all of which are incorporated herein by reference.

Some aspects herein regard a method of styling hair. Such a method can comprise, for example, at least steps (a) and (b), or steps (c) and (d), as follows:
(a) contacting (e.g., coating) hair with a composition comprising an alpha-glucan ether derivative herein, thereby providing treated hair (or coated hair), and
(b) putting the treated hair (or the coated hair) into a desired form; or
(c) putting hair into a desired form, and
(d) contacting (e.g., coating) the hair of step (c) with a composition comprising an alpha-glucan ether derivative herein, thereby providing treated hair (or coated hair); and
(e) optionally, removing solvent, if present, that was used to deliver the alpha-glucan ether derivative to the hair in step (a) or (d).

Such a method can optionally be characterized as a hair styling method. Contacting in a hair styling method can be performed, for example, by applying/treating hair with a hairstyling composition herein (e.g., gel, mouse, spray) comprising at least one alpha-glucan ether derivative. Hair to be treated in a hair styling method, particularly in steps (a) or (d), typically can be wet or dry. Step (e) of removing solvent can be performed by drying, for example, such as by a drying method disclosed herein (e.g., air drying or blow drying, with either room temperature or heated air). Drying can be done with (or without) agitation of the treated hair, such as by combing or brushing while drying. Optionally, a styling method herein can comprise, after step (b) or step (d) (but before optional step [e]), a step of applying steam to the treated hair. Step (b) or (c) of putting hair into a desired form can be performed in some aspects by straightening, curling, or otherwise putting the hair into a form that is different from the form the hair was in as it existed before step (a), (b), or (c). Hair that is styled by a styling method herein can hold, optionally without the need to apply any device and/or further material to the styled hair (i.e., while in a free-standing state), the desired form for a period of at least 1, 2, 3, 4, 5, or more days, for example. Such style retention can be in conditions of dry air (e.g., relative humidity s 50%) or humid air (e.g., relative humidity over 50%), for example (typically for a period of time during which the styled hair is not washed or rinsed).

Non-limiting examples of compositions and methods disclosed herein include:

1. A composition comprising at least a solvent and an ether derivative of an alpha-glucan, wherein (i) at least about 40% of the glycosidic linkages of the alpha-glucan are alpha-1,6 linkages, (ii) the alpha-glucan has a weight-average molecular weight (Mw) of about 1 kDa to about 2000 kDa, (iii) the alpha-glucan has a degree of substitution (DoS) of about 0.01 to about 3.0 with at least one positively charged organic group that is ether-linked to the alpha-glucan, and (iv) the solvent comprises water and at least about 40% (v/v) of a polar organic solvent; wherein the ether derivative is dissolved and/or dispersed in the solvent.
2. The composition of embodiment 1, wherein the polar organic solvent is an alcohol, optionally wherein the alcohol is ethanol.
3. The composition of embodiment 1 or 2, wherein the composition comprises at least about 50% (v/v) of the polar organic solvent, optionally wherein the composition comprises at least about 67% (v/v) or 80% (v/v) of the polar organic solvent.
4. The composition of embodiment 1, 2, or 3, wherein the composition comprises at least about 0.1 wt % of the ether derivative, optionally wherein the composition comprises about 0.1 wt % to about 10% wt % of the ether derivative.
5. The composition of embodiment 1, 2, 3, or 4, wherein at least about 30% of the ether derivative by weight is dissolved and/or dispersed (stably dispersed) in the solvent.
6. The composition of embodiment 1, 2, 3, 4, or 5, wherein the composition has a turbidity of less than 200 NTU (nephelometric turbidity units), optionally wherein the composition has a turbidity of less than 20 NTU.
7. The composition of embodiment 1, 2, 3, 4, 5, or 6, wherein at least about 90% of the glycosidic linkages of the alpha-glucan are alpha-1,6 linkages.
8. The composition of embodiment 1, 2, 3, 4, 5, 6, or 7, wherein the alpha-glucan comprises at least 1% (optionally at least 5%) alpha-1,2 and/or alpha-1,3 branches.
9. The composition of embodiment 1, 2, 3, 4, 5, 6, 7, or 8, wherein the alpha-glucan has an Mw of about 1 kDa to about 500 kDa.
10. The composition of embodiment 1, 2, 3, 4, 5, 6, 7, 8, or 9, wherein the DoS is at least about 0.05, optionally wherein the DoS is at least about 0.1 or at least about 0.4.
11. The composition of embodiment 1, 2, 3, 4, 5, 6, 7, 8, 9, or 10, wherein the positively charged organic group comprises a substituted ammonium group.
12. The composition of embodiment 11, wherein the substituted ammonium group comprises a quaternary ammonium group.
13. The composition of embodiment 12, wherein the quaternary ammonium group comprises: (a) three C₁-C₄ alkyl groups (e.g., three methyl groups), or (b) two C₁-C₄ alkyl groups (e.g., two methyl groups) and one C₄ to C₂₀ alkyl group (e.g., a C₅-C₂₀, C₈-C₁₆, C₁₀-C₁₄, or C₁₂ alkyl group).
14. The composition of embodiment 1, 2, 3, 4, 5, 6, 7, 8, 9, 10, 11, 12, or 13, wherein the composition is a household care product, personal care product, industrial product, ingestible product (e.g., food product), or pharmaceutical product.
15. The composition of embodiment 1, 2, 3, 4, 5, 6, 7, 8, 9, 10, 11, 12, 13, or 14, wherein the composition is a hair care product.
16. The composition of embodiment 15, wherein the hair care product is a hair styling product, optionally wherein the hair styling product is a spray, gel/lotion, or mousse/foam.
17. A method of producing a composition according to embodiment 1, 2, 3, 4, 5, 6, 7, 8, 9, 10, 11, 12, 13, 14, 15, or 16, the method comprising: mixing the solvent and the ether derivative together, thereby producing the composition.
18. A method of producing a film or coating, the method comprising: (a) providing a composition according to embodiment 1, 2, 3, 4, 5, 6, 7, 8, 9, 10, 11, 12, 13, 14, 15, or 16, (b) contacting the composition with a surface (e.g., hair surface), and (c) removing at least about 95% by weight of the solvent to form a film or coating on the surface.
19. A film or coating comprising at least one ether derivative of an alpha-glucan as described in embodiment 1, 7, 8, 9, 10, 11, 12, or 13, optionally wherein the film or coating is on the surface of a material (e.g., hair) as disclosed herein.
20. A method of styling hair, the method comprising at least steps (a) and (b), or steps (c) and (d), as follows: (a) contacting hair with a composition according to embodiment 1, 2, 3, 4, 5, 6, 7, 8, 9, 10, 11, 12, 13, 14, 15, or 16, thereby providing treated hair, and (b) putting the treated hair (or the coated hair) into a desired form; or (c) putting hair into a desired form, and (d) contacting the hair of step (c) with a composition according to embodiment 1, 2, 3, 4, 5, 6, 7, 8, 9, 10, 11, 12, 13, 14, 15, or 16, thereby providing treated hair; and (e) optionally, removing the solvent from the composition that was contacted with the hair in step (a) or (d).

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Materials/Methods

Unless otherwise stated, all ingredients are available from Sigma-Aldrich, St. Louis, Missouri and were used as received. 3-chloro-2-hydroxypropyltrimethylammonium chloride (QUAB 188), glycidyltrimethylammonium chloride (also referred to as 2,3-epoxypropyltrimethylammonium chloride) (QUAB 151), and 3-chloro-2-hydroxypropyl dodecyldimethylammonium chloride (QUAB 342) were obtained from SKW QUAB Chemicals.

### Method for Determining Anomeric Linkages by NMR Spectroscopy

Glycosidic linkages in water soluble oligosaccharides and polysaccharide products synthesized by a glucosyltransferase GTF8117 and alpha-1,2 branching enzyme were determined by ¹H NMR (nuclear magnetic resonance) spectroscopy. Dry oligosaccharide/polysaccharide polymer (6 mg to 8 mg) was dissolved in a solution of 0.7 mL of 1 mM DSS (4,4-dimethyl-4-silapentane-1-sulfonic acid; NMR reference standard) in D₂O. The sample was stirred at ambient temperature overnight. 525 μL of the clear homogeneous solution was transferred to a 5-mm NMR tube. 2D ¹H, ¹³C homo/hetero-nuclear suite of NMR experiments were used to identify AGU (anhydroglucose unit) linkages. The data were collected at 20° C. and processed on a Bruker Avance III NMR spectrometer, operating at either 500 MHz or 600 MHz. The systems are equipped with a proton-optimized, helium-cooled cryoprobe. The 1D ¹H NMR spectrum was used to quantify glycosidic linkage distribution and found the polysaccharide backbone as primarily alpha-1,6. The results reflect the ratio of the integrated intensity of an NMR resonance representing an individual linkage type divided by the integrated intensity of the sum of all peaks which represent glucose linkages, multiplied by 100.

### ¹H Nuclear Magnetic Resonance (NMR) Method for Determining Molar Substitution of Alpha-11,6-Glucan Ether Derivatives

Approximately 30 mg of alpha-1,6-glucan ether derivative was weighed into a vial on an analytical balance. The vial was removed from the balance and 1.0 mL of deuterium oxide was added to the vial. A magnetic stir bar was added to the vial and the mixture was stirred to suspend the solid. Deuterated sulfuric acid (50% v/v in D₂O), 1.0 mL, was then added to the vial and the mixture was heated at 90° C. for 1 hour to depolymerize and solubilize the polymer. The solution was allowed to cool to room temperature and then a 0.8-mL portion of the solution was transferred into a 5-mm NMR tube using a glass pipet. A quantitative ¹H NMR spectrum was acquired using an Agilent VNMRS 400 MHz NMR spectrometer equipped with a 5-mm Autoswitchable Quad probe. The spectrum was acquired at a spectral frequency of 399.945 MHz, using a spectral window of 6410.3 Hz, an acquisition time of 3.744 seconds, an inter-pulse delay of 10 seconds and 64 pulses. The time domain data were transformed using exponential multiplication of 0.50 Hz.

Determination of Weight-Average Molecular Weight and/or Degree of Polymerization

Degree of polymerization (DP) was determined by size-exclusion chromatography (SEC). For SEC analysis, dry alpha-1,6-glucan ether derivative was dissolved in phosphate-buffered saline (PBS) (0.02-0.2 mg/mL). The chromatographic system used was an Alliance™ 2695 liquid chromatograph from Waters Corporation (Milford, MA) coupled with three on-line detectors: a differential refractometer 410 from Waters, a multi-angle light-scattering photometer Heleos™ 8+ from Wyatt Technologies (Santa Barbara, CA), and a differential capillary viscometer ViscoStar™ from Wyatt Technologies. The columns used for SEC were two Tosoh Haas Bioscience TSK GMPW_{XL}g3K and g4K G3000PW and G4000PW polymeric columns for aqueous polymers. The mobile phase was PBS. The chromatographic conditions used were 30° C. at column and detector compartments, 30° C. at sample and injector compartments, a flow rate of 0.5 mL/min, and injection volume of 100 μL. The software packages used for data reduction were Astra version 6 from Wyatt (triple detection method with column calibration).

Representative Preparation of Alpha-1,6-Glucan with Alpha-1,2 Branching

Except as otherwise noted, all alpha-1,6-glucans in the Examples contained a linear backbone with 100% alpha-1,6 glycosidic linkages. Alpha-1,2 branches (i.e., pendant alpha-1,2-linked glucose groups) were added to such backbones. Except as otherwise noted, all reported molecular weights are weight-average and regard the alpha-1,6-glucan backbone prior to branching.

Methods to prepare alpha-1,6-glucan containing various amounts of alpha-1,2 branching are disclosed in U.S. Appl. Publ. No. 2018/0282385, which is incorporated herein by reference. Reaction parameters such as sucrose concentration, temperature, and pH can be adjusted to provide alpha-1,6-glucan having various levels of alpha-1,2-branching and molecular weight. A representative procedure for the preparation of alpha-1,2-branched alpha-1,6-glucan is provided below (containing 19% alpha-1,2-branching and 81% alpha-1,6 linkages). The 1 D ¹H-NMR spectrum was used to quantify glycosidic linkage distribution. Additional samples of alpha-1,6-glucan with alpha-1,2-branching were prepared similarly. For example, one sample contained 32% alpha-1,2-branching and 68% alpha-1,6 linkages, and another contained 10% alpha-1,2-branching and 90% alpha-1,6 linkages.

Soluble alpha-1,6-glucan with about 19% alpha-1,2 branching was prepared using stepwise combination of glucosyltransferase (dextransucrase) GTF8117 and alpha-1,2 branching enzyme GTFJ18T1, according to the following procedure. A reaction mixture (2 L) comprised of sucrose (450 g/L), GTF8117 (9.4 U/mL), and 50 mM sodium acetate was adjusted to pH 5.5 and stirred at 47° C. Aliquots (0.2-1 mL) were withdrawn at predetermined times and quenched by heating at 90° C. for 15 minutes. The resulting heat-treated aliquots were passed through a 0.45-μm filter.

The flow-through was analyzed by HPLC to determine the concentration of sucrose, glucose, fructose, leucrose, oligosaccharides and polysaccharides. After 23.5 hours, the reaction mixture was heated to 90° C. for 30 minutes. An aliquot of the heat-treated reaction mixture was passed through a 0.45-μm filter and the flow-through was analyzed for soluble mono/disaccharides, oligosaccharides, and polysaccharides. A major product was linear dextran with a DPw of 93 (100% alpha-1,6 glycosidic linkages).

A second reaction mixture was prepared by adding 238.2 g of sucrose and 210 mL of alpha-1,2-branching enzyme GTFJ18T1 (5.0 U/mL) to the leftover heat-treated reaction mixture that was obtained from the GTF8117 reaction described immediately above. The mixture was stirred at 30° C. with a volume of ˜2.2 L. Aliquots (0.2-1 mL) were withdrawn at predetermined times and quenched by heating at 90° C. for 15 minutes. The resulting heat-treated aliquots were passed through a 0.45-μm filter. The flow-through was analyzed by HPLC to determine the concentration of sucrose, glucose, fructose, leucrose, oligosaccharides and polysaccharides. After 95 hours, the reaction mixture was heated to 90° C. for 30 minutes. An aliquot of the heat-treated reaction mixture was passed through a 0.45-μm filter and the flow-through was analyzed for soluble mono/disaccharides, oligosaccharides, and polysaccharides. Leftover heat-treated mixture was centrifuged using 1-L centrifugation bottles. The supernatant was collected and cleaned more than 200-fold using an ultrafiltration system with 1- or 5-kDa MWCO cassettes and deionized water. The cleaned oligo/polysaccharide product solution was dried. Dry sample was then analyzed by ¹H-NMR spectroscopy to determine the anomeric linkages of the oligosaccharides and polysaccharides.

### Example 1

This Example describes preparation of a quaternary ammonium alpha-1,6-glucan ether compound, specifically trimethylammonium hydroxypropyl alpha-1,6-glucan.

Polysaccharide solution (43% solids, 7.3 kg; alpha-1,6-glucan with 32% alpha-1,2-branching and 68% alpha 1,6 linkages, Mw 53 kDa) was charged into a 22 L reactor equipped with an overhead stirrer. To the stirring solution was added 2.72 kg of 50% NaOH solution. The mixture was heated to 50° C. To this was added 7.6 kg of a 65% solution of 3-chloro-2-hydroxypropyltrimethylammonium chloride (QUAB 188) with an addition funnel over 2 hours and 45 min. The reaction was then kept at 58° C. for 3 hours. The reaction was diluted with water (500 mL), and neutralized with 18 wt % HCl. The product was purified by ultrafiltration (10-kDa membrane), and freeze-dried. The degree of substitution of the product was determined to be 0.4 by ¹H NMR.

### Example 2

This Example describes preparation of a quaternary ammonium alpha-1,6-glucan ether compound, specifically trimethylammonium hydroxypropyl alpha-1,6-glucan.

To a 1-L round bottom flask equipped with an overhead stirrer was added 100 mL water, followed by 100 g of polysaccharide (alpha-1,6-glucan with 10% alpha-1,2-branching and 90% alpha 1,6 linkages, Mw 60 kDa). After dissolution, 50% sodium hydroxide solution was added (87 g) over 5-10 min. The mixture was stirred at room temperature for 1 hour. To this was added 265 g of a 60% solution of 3-chloro-2-hydroxypropyltrimethylammonium chloride (QUAB 188) over an additional 10 min. The mixture was heated at 60° C. under nitrogen for 3 hours. The mixture was cooled to about 50° C., and neutralized with 18% HCl. The resulting solution was diluted with water (4 L) and the product was purified by ultrafiltration (30-kDa membrane), and freeze dried. The degree of substitution of the product was determined to be 0.6 by ¹H NMR.

### Example 3

This Example describes preparation of a quaternary ammonium alpha-1,6-glucan ether compound, specifically trimethylammonium hydroxypropyl alpha-1,6-glucan.

To a 2-L reactor equipped with an overhead stirrer was added 690 g of a polysaccharide solution (29% solids; alpha-1,6-glucan with 5% alpha-1,2-branching and 95% alpha 1,6 linkages, Mw 185 kDa). The solution was stirred. To this stirring solution was added 12 g of 50% sodium hydroxide dropwise. The mixture was stirred at room temperature for 45 min. To this stirring mixture was added 100 g 71-75% solution of glycidyltrimethylammonium chloride (QUAB 151). The mixture was heated for 4 hours at 60° C. The mixture was diluted with 200 mL water, and neutralized with 18 wt % HCl. The product was purified by ultrafiltration (30-kDa membrane), and freeze-dried. The degree of substitution of the product was determined to be 0.4 by ¹H NMR.

### Example 4

This Example describes preparation of a quaternary ammonium alpha-1,6-glucan ether compound, specifically trimethylammonium hydroxypropyl alpha-1,6-glucan.

To a 2-L reactor equipped with an overhead stirrer was added 690 g of a polymer solution (29% solids; alpha-1,6-glucan with 5% alpha-1,2-branching and 95% alpha 1,6 linkages, Mw 185 kDa). The solution was stirred. To this stirring solution was added 12 g of 50% sodium hydroxide dropwise. The mixture was stirred at room temperature for 45 min. To this stirring mixture was added 33 g 71-75% solution of glycidyltrimethylammonium chloride (QUAB 151). The mixture was heated for 4 hours at 60° C. The mixture was diluted with 200 mL water, and neutralized with 18 wt % HCl. The product was purified by ultrafiltration (30-kDa membrane), and freeze-dried. The degree of substitution of the product was determined to be 0.03 by ¹H NMR.

### Example 5

This Example describes preparation of a quaternary ammonium alpha-1,6-glucan ether compound, specifically dodecyldimethylammonium hydroxypropyl alpha-1,6-glucan.

A 4-neck, 500-mL reactor equipped with a mechanical stir rod, thermocouple, and addition funnel was charged with 19 g of water. Polysaccharide (21 g, alpha-1,6-glucan with 32% alpha-1,2-branching and 68% alpha 1,6 linkages, Mw 68 kDa) was then added to provide a solution. The solution was stirred while 137 g of 40 wt % 3-chloro-2-hydroxypropyl dodecyldimethylammonium chloride (QUAB 342) was added thereto. The resulting mixture was stirred at room temperature for 2 hours. Sodium hydroxide (15.8 g, 50 wt %) was added over a 10-minute period. The reaction mixture was heated to 60° C. (10 min) and stirred at 57-60° C. for 3 hours. After being cooled to 35° C., the reaction mixture was poured into water to a total volume about 3 L. The pH of the mixture was adjusted to about 7 by the addition of 18.5 wt % hydrochloric acid. The product was purified by using ultrafiltration (5-kDa membrane) and freeze-dried. The degree of substitution of the product was determined to be 0.4 by ¹H NMR.

### Example 6

This Example describes preparation of a quaternary ammonium alpha-1,6-glucan ether compound, specifically dodecyldimethylammonium hydroxypropyl alpha-1,6-glucan.

A 4-neck, 500-mL reactor equipped with a mechanical stir rod, thermocouple, and addition funnel was charged with 80 g of a 3-chloro-2-hydroxypropyl dodecyldimethylammonium chloride (QUAB 342) preparation containing 32 g of the chloride and 48 g water. Glucan powder (21 g, alpha-1,6-glucan with 32% alpha-1,2-branching and 68% alpha 1,6 linkages, Mw 68 kDa) was then added. The mixture was stirred at room temperature for 2 hours. Sodium hydroxide (10 g, 50 wt %) was added over a 10-minute period. Water (10 mL) was then added. The reaction mixture was heated to 60° C. (10 min) and stirred at 58-60° C. for 3 hours. After being cooled to 35° C., the reaction mixture was poured into water to a total volume of about 3 L. The pH of the mixture was adjusted to about 7 by the addition of 18.5 wt % HCl. The mixture was filtered and no solid was observed in the filter. The filtrate was purified by ultra-filtration (10K membrane), and then freeze-dried to render a product. The degree of substitution of the product was determined to be 0.4 by ¹H NMR.

### Example 7

This Example describes various quaternary ammonium alpha-1,6-glucan ether compounds produced according to the presently disclosed procedures. In the compounds listed in Table 1 below, the cationic group is a quaternary ammonium group substituted with three methyl groups (i.e., trimethyl ammonium), unless otherwise indicated with one asterisk (*). The quaternary ammonium group in each compound is linked to the ether group (and thus to the glucan backbone) by a hydroxypropyl group, but any suitable alkyl group or other hydroxyalkyl group could be used to link, accordingly.

TABLE 1

| | Alpha-1,6-Glucan Cationic Ether | | |
|---|---|---|---|
| | Backbone | | Degree of Alpha- |
| Polymer | MW (kDa) | DoS | 1,2 Branching |
| A | 40 | 0.5 | 40% |
| B | 40 | 0.5 | 40% |
| | (75)** | | |
| C | 17 | 0.3 | 40% |
| D | 40 | 0.4* | 40% |
| | (59)** | | |
| E | 40 | 0.26* | 40% |
| F | 40 | 0.8 | 40% |
| | (84)** | | |
| G | 109 | 0.51 | 26% |
| | (148)** | | |
| H | 194 | 0.50 | 41% |
| | (245)** | | |
| I | 194 | 0.7 | 41% |
| | (269)** | | |
| J | 185 | 0.15 | 5% |
| K | 185 | 0.38 | 5% |
| L | 185 | 0.03 | 5% |
| M | 200 | 0.21 | 20% |
| N | 200 | 0.19 | 10% |
| O | 185 | 0.05 | 5% |
| P | 185 | 0.40 | 20% |
| Q | 185 | 0.07 | 5% |
| R | 185 | 0.11 | 5% |
| S | 185 | 0.59 | 5% |
| T | 109 | 0.22 | 26% |

*Cationic group: quaternary ammonium group substituted with two methyl groups and one C₁₂ alkyl group (dimethyl, C₁₂ ammonium group).
**Parenthetical number is molecular weight of ether compound (i.e., backbone plus derivatized cationic ether groups).

### Example 8

This Example describes testing whether alpha-1,2-branched alpha-1,6-glucan cationic ether compounds are capable of dissolving and/or dispersing in solvents having an elevated polar organic solvent content. In particular, glucan derivatives of the present disclosure with hydroxypropyl trimethylammonium ether groups were tested for solvation and/or dispersion activity in aqueous ethanol solutions. Some glucan ether derivatives were identified to have such activity and thus are suitable candidates for use in application formulations having a high alcohol concentration (e.g., >=40% v/v), for example.

Solubility test: The cationic glucan ethers used in this study are listed below in Table 2. Each glucan ether sample was first fully dissolved in water. Various amounts of ethanol were then added to this solution to prepare individual preparations containing 1 wt % of glucan ether compound and 56%, 67%, 75%, 80%, or 90% (v/v) ethanol; this mixing with ethanol was conducted within about 2 minutes. These mixing steps were conducted at room temperature. The turbidity of each preparation was measured in NTU (nephelometric turbidity units) using a calibrated turbidimeter (Hach 2100AN) 24 hours after producing each preparation. A low turbidity readout for a sample indicated that the sample could tolerate being in an aqueous ethanol-containing solvent by mostly or completely dissolving and/or uniformly dispersing therein.

TABLE 2

| Cationic Alpha-1,6-Glucan | | | Turbidity (NTU) of Ethanol/Water | | | | |
|---|---|---|---|---|---|---|---|
| Ether Compound^{a} | | | Solution with Compound Added | | | | |
| Mw (kDa) | % Branch. | | % Ethanol (v/v) | | | | |
| (+/−10%)^{b} | (Alpha-1,2) | DoS | 56% | 67% | 75% | 80% | 90% |
| 300 | 36 | 0.05 | 1 | 410 | x^{c} | x | x |
| | 5 | 0.06 | 2 | 214 | x | x | x |
| | | 0.03 | 534 | x | x | x | x |
| 200 | 5 | 0.4 | 1 | 1 | 114 | 306 | x |
| | | 0.2 | 654 | x | x | x | x |
| | | 0.07 | 1204 | x | x | x | x |
| | 20 | 0.4 | 1 | 1 | 74 | 150 | x |
| | | 0.05 | 1 | 328 | x | x | x |
| 50 | 37 | 0.82 | 1 | 1 | 1 | 3 | 53 |
| | | 0.6 | 1 | 2 | 3 | 134 | x |
| | | 0.44 | 2 | 2 | 2 | 289 | x |
| 20 | 37 | 0.17 | 2 | 3 | 5615 | x | x |
| | | 0.07 | 3 | 120 | x | x | x |

^{a}Alpha-1,2-branched alpha-1,6-glucan substituted with hydroxypropyl trimethylammonium ether groups. The degree of substitution (DoS) with ether groups and percent alpha-1,2 branching for each glucan ether derivative are listed.
^{b}Weight-average molecular weight of the alpha-1,6-glucan backbone prior to alpha-1,2-branching and ether derivatization.
^{c}Each “x” mark denotes that the preparation resulting from mixing the compound in the listed ethanol/water solution was not uniform (e.g., phase-separated) and could not be qualified for turbidity testing.

In general, preparations with an NTU below 20 (Table 2) appeared to be very transparent. It is generally contemplated that preparations with an NTU value below 20 (as tested above) are acceptable for most products and applications that benefit from having high transparency. NTU values below 200 (Table 2) were generally associated with high compound dispersibility and/or substantial solubility. Though the tested compounds (Table 2) exhibited various abilities to tolerate being in ethanol-containing aqueous solutions (e.g., some compounds remained dispersed or dissolved under higher ethanol concentrations compared to other compounds), all the compounds were found to tolerate being in aqueous solutions having the elevated base ethanol concentration of 56% v/v, which suggests that they have some degree of ethanol tolerance. Compounds exhibiting an NTU below 200 have the potential to be used at a concentration of 1 wt % or higher in ethanol-containing aqueous applications. Compounds with higher NTU values (e.g., the compound with an NTU of 1204 NTU in 56% v/v ethanol), could still have applicable dispersibility, being acceptable if used at a lower concentration (e.g., 0.1 wt %).

The data in Table 2 indicate that, in general, the DoS of a cationic alpha-glucan ether derivative herein has a significant effect on the solubility or dispersibility of the derivative in ethanol-containing aqueous solutions. For example, a DoS of 0.4 or higher provided compounds with the ability to completely or mostly dissolve in an aqueous solvent having at least 80% (v/v) ethanol (Table 2). The alpha-glucan ether having a DoS of 0.82 (Table 2) had the best solubility profile, even being mostly soluble in aqueous solvent have 90% (v/v) ethanol.

An additional study was performed in a manner similar to the above process. A cationic alpha-1,6-glucan ether derivative (the same compound as listed in Table 2 with 200 kDa backbone Mw, 20% alpha-1,2 branching, 0.05 DoS) was dissolved in water, after which various amounts of ethanol were mixed in portions into the solution over an average period of about 4 hours (i.e., ethanol added at a slow rate) to prepare individual preparations containing 1 wt % of glucan ether compound and 56%, 67%, 75%, 80%, 90%, or 95% (v/v) ethanol. Interestingly, this process led to formation of more stable preparations as reflected by the following turbidity measurements (ethanol % v/v—NTU): 56% —<1 NTU, 67% —116 NTU, 75% —26 NTU, 80% —35 NTU, 90% —110 NTU (95%—phase-separated, turbidity not measurable). It is believed that applying this slower alcohol mixing regimen with other alpha-glucan ether derivatives herein will likewise yield more favorable results of stable solutions and dispersions in high alcohol concentration compositions.

An example of a gradual, portion-wise alcohol mixing protocol was as follows: cationic alpha-1,6-glucan ether herein (200 mg) was weighed into a glass vial and fully dissolved in DI-water (4 mL), after which ethanol (4 mL, 200 proof) was added. The resulting preparation was stirred vigorously for at least 30 minutes. A second portion of ethanol (4 mL, 200 proof) was then added followed by at least 30 minutes of vigorous stirring. This procedure of adding ethanol and stirring was repeated until there was a total of 20 gram of preparation, which contained 1 wt % glucan ether compound and 80% (v/v) ethanol.

Film quality test: Each preparation used in Table 2 of the above solubility study was poured into a Petri dish and allowed to dry overnight at room temperature to form a film, which was then examined for quality. Significantly, all the films were clear and coherent.

The combination of good solubility (low turbidity) and high quality film formation (e.g., ability to form clear film) render the tested compounds as being useful in various applications. Thus, for example, it is contemplated that compounds herein can be employed in hair styling products that benefit from being able to be applied to hair in a clear and transparent manner to provide styling hold while avoiding an unclean look.

Hair curl retention test: Compounds were prepared for analysis in a similar manner as above for the solubility test. Briefly, preparations were produced having a glucan ether compound (at 1, 2 or 4 wt %) in aqueous solutions containing 50% or 67% (v/v) ethanol. ˜0.5 gram of each compound preparation (Table 3) was individually applied to a hair tress (8″ RINBOOOL hair swatches). The resulting hair tress was dried at room temperature overnight with the second half of the hair tress curled back in a >90 degree angle. Each hair tress was then hung in a 45° C. oven and heated for 3 hours, after which the hair tress was combed through once with a 4-tooth comb. The height of the curled half of each hair tress was then measured and compared to the height of the tress as it existed before the hanging/combing (Table 3). In a negative control experiment (application of ethanol/water solution with no added compound), the height of the curled half of the hair tress changed by more than 9.0 cm (Table 3). A small change in height measurement indicates that a compound can be used for hair styling retention.

TABLE 3

| | Hair Tress |
|---|---|
| Compound^{a} | Height Change^{b} |
| Preparation | (cm) |
| Negative Control | >9.0 |
| 200 kDa^{c}, 5%^{d}, DoS 0.07 | 1.6 |
| 1 wt % in 50/50 ethanol/ | |
| water solution | |
| 20 kDa, 37%, DoS 0.17 | 5.4 |
| 1 wt % in 67/33 ethanol/ | |
| water solution | |
| 20 kDa, 37%, DoS 0.17 | 2.5 |
| 2 wt % in 67/33 ethanol/ | |
| water solution | |
| 20 kDa, 37%, DoS 0.17 | |
| 4 wt % in 50/50 ethanol/ | 1.5 |
| water solution | |
| 20 kDa, 37%, DoS 0.07 | |
| 1 wt % in 50/50 ethanol/ | 4.3 |
| water solution | |
| 200 kDa, 20%, DoS 0.40 | 2.0 |
| 4 wt % in 50/50 ethanol/ | |
| water solution | |
| 200 kDa, 20%, DoS 0.05 | |
| 1 wt % in 50/50 ethanol/ | 1.5 |
| water solution | |
| 200 kDa, 20%, DoS 0.05 | |
| 2 wt % in 50/50 ethanol/ | 1.0 |
| water solution | |
| 200 kDa, 20%, DoS 0.05 | |
| 4 wt % in 50/50 ethanol/ | 0.9 |
| water solution | |

^{a}Alpha-1,2-branched alpha-1,6-glucan substituted with hydroxypropyl trimethylammonium ether groups. The degree of substitution (DoS) with ether groups and percent alpha-1,2 branching for each glucan ether derivative are listed.
^{b}Hair tress height change following styling with sample preparation. A small height change reflects effective hair styling (i.e., applied alpha-glucan derivative maintains bend in hair tress).
^{c}Weight-average molecular weight of the alpha-1,6-glucan backbone prior to alpha-1,2-branching and ether derivatization (applies to each listed compound).
^{d}Percent alpha-1,2 branching of the alpha-1,6-glucan (applies to each listed compound).

The data in Table 3 indicate that cationic alpha-glucan ether compounds herein are effective at holding hair in a styled position.

### Example 9

This Example describes additional testing of whether alpha-1,6-glucan cationic ether compounds are capable of dissolving and/or dispersing in solvents having an elevated polar organic solvent content. The alpha-glucan compounds of this study were derivatized with hydroxypropyl lauryldimethylammonium ether groups (i.e., dodecyldimethylammonium hydroxypropyl alpha-glucan ethers were used); each compound is listed in Table 4.

Each alpha-glucan ether sample was first fully dissolved in water. Various amounts of ethanol were then added in portions to this solution to prepare individual preparations containing 1.5 wt % of alpha-glucan ether compound and 50%, 67%, 75%, 80%, or 90% (v/v) ethanol; this mixing with ethanol was conducted for at least 30 minutes. These mixing steps were conducted at room temperature. The turbidity of each preparation was measured in NTU using a calibrated turbidimeter (Hach 2100 Q) 24 hours after producing each preparation. A low turbidity readout for a sample indicated that the sample could tolerate being in an aqueous ethanol-containing solvent by mostly or completely dissolving and/or uniformly dispersing therein.

TABLE 4

| | | | | Turbidity (NTU) of | | | | |
|---|---|---|---|---|---|---|---|---|
| Cationic Alpha-1,6-Glucan | | | | Ethanol/Water Solution with | | | | |
| Ether Compound^{a} | | | | Compound Added | | | | |
| Mw | | % Branch. | | % Ethanol (v/v) | | | | |
| (+/−10%)^{b} | | (Alpha-1,2) | DoS | 50% | 67% | 75% | 80% | 90% |
| 200 | kDa | 5 | 0.09 | 4 | 4 | 7 | 10 | 83 |
| 200 | kDa | 5 | 0.06 | 5 | 12 | 18 | 23 | 53 |
| 300 | kDa | 5 | 0.05 | 7 | 15 | 26 | 37 | 178 |
| 100 | MDa^{d} | ND | 0.05 | 272 | x^{c} | x | x | x |

^{a}Alpha-1,2-branched alpha-1,6-glucan substituted with hydroxypropyl dodecyldimethylammonium ether groups. The degree of substitution (DoS) with ether groups and percent alpha-1,2 branching for each glucan ether derivative are listed.
^{b}Weight-average molecular weight of the alpha-1,6-glucan backbone prior to alpha-1,2-branching (first three rows) and ether derivatization. The 100 MDa glucan was not subjected to an alpha-1,2 branching step prior to etherification.
^{c}Each “x” mark denotes that the preparation resulting from mixing the compound in the listed ethanol/water solution was not uniform (e.g., phase-separated) and could not be qualified for turbidity testing.
^{d}Alpha-1,6-glucan as produced using GTF 0768 as described in U.S. Patent Appl. Publ. No. 2016/0122445 (incorporated herein by reference). The GTF reaction initially comprised 100 g/L sucrose. Percent alpha-1,2 branching was not determined (ND).

In general, preparations with an NTU below 20 (Table 4) appeared to be very transparent. NTU values below 200 (Table 4) were generally associated with high compound dispersibility and/or substantial solubility. Though the tested compounds (Table 4) exhibited various abilities to tolerate being in ethanol-containing aqueous solutions (e.g., some compounds remained dispersed or dissolved under higher ethanol concentrations compared to other compounds), all except one of the compounds were found to tolerate being in aqueous solutions having the elevated base ethanol concentration of 90% v/v, which suggests that they have some degree of ethanol tolerance. Compounds exhibiting an NTU below 200 have the potential to be used at a concentration of 1.5 wt % or higher in ethanol-containing aqueous applications. Compounds with higher NTU values (e.g., the compound with an NTU of 272 NTU in 50% v/v ethanol), potentially could have applicable dispersibility, being acceptable if used at a lower concentration (e.g., 0.1 wt %).

What is claimed is:

1. A composition comprising at least a solvent and an ether derivative of an alpha-glucan, wherein
(i) at least about 40% of the glycosidic linkages of the alpha-glucan are alpha-1,6 linkages,
(ii) the alpha-glucan has a weight-average molecular weight (Mw) of about 1 kDa to about 2000 kDa,
(iii) the alpha-glucan has a degree of substitution (DoS) of about 0.01 to about 3.0 with at least one positively charged organic group that is ether-linked to the alpha-glucan, and
(iv) the solvent comprises water and at least about 40% (v/v) of a polar organic solvent;
wherein the ether derivative is dissolved and/or dispersed in the solvent.

2. The composition of claim 1, wherein the polar organic solvent is an alcohol, optionally wherein the alcohol is ethanol.

3. The composition of claim 1, wherein the composition comprises at least about 50% (v/v) of the polar organic solvent, optionally wherein the composition comprises at least about 67% (v/v) of the polar organic solvent.

4. The composition of claim 1, wherein the composition comprises at least about 0.1 wt % of the ether derivative, optionally wherein the composition comprises about 0.1 wt % to about 10% wt % of the ether derivative.

5. The composition of claim 1, wherein at least about 30% of the ether derivative by weight is dissolved and/or dispersed in the solvent.

6. The composition of claim 1, wherein the composition has a turbidity of less than 200 NTU (nephelometric turbidity units), optionally wherein the composition has a turbidity of less than 20 NTU.

7. The composition of claim 1, wherein at least about 90% of the glycosidic linkages of the alpha-glucan are alpha-1,6 linkages.

8. The composition of claim 1, wherein the alpha-glucan comprises at least 1% alpha-1,2 and/or alpha-1,3 branches.

9. The composition of claim 1, wherein the alpha-glucan has an Mw of about 1 kDa to about 500 kDa.

10. The composition of claim 1, wherein the DoS is at least about 0.02, optionally wherein the DoS is at least about 0.05.

11. The composition of claim 1, wherein the positively charged organic group comprises a substituted ammonium group.

12. The composition of claim 11, wherein the substituted ammonium group comprises a quaternary ammonium group.

13. The composition of claim 12, wherein the quaternary ammonium group comprises:
(a) three C₁-C₄ alkyl groups, or
(b) two C₁-C₄ alkyl groups and one C₄ to C₂₀ alkyl group.

14. The composition of claim 1, wherein the composition is a household care product, personal care product, industrial product, ingestible product (e.g., food product), or pharmaceutical product.

15. The composition of claim 1, wherein the composition is a hair care product.

16. The composition of claim 15, wherein the hair care product is a hair styling product, optionally wherein the hair styling product is a spray, gel/lotion, or mousse/foam.

17. A method of producing a composition according to claim 1, said method comprising:
mixing the solvent and the ether derivative together, thereby producing the composition.

18. A method of producing a film or coating, the method comprising:
(a) providing a composition according to claim 1,
(b) contacting the composition with a surface, and
(c) removing at least about 95% by weight of the solvent to form a film or coating on the surface.
